(19) United States
(12) Patent Application Publication
(10) Pub. No.: US 2014/0227690 A1
(43) Pub. Date: Aug. 14, 2014

(54) METHODS AND COMPOSITIONS FOR ASSESSMENT OF FETAL LUNG MATURITY

(71) Applicant: St. John Heallth, Warren, MI (US)

(72) Inventors: Robert Alan Welch, Canton, MI (US); Michael Kenneth Shaw, Northville, MI (US)

(73) Assignee: St. John Heallth, Warren, MI (US)

(21) Appl. No.: 13/838,592

(22) Filed: Mar. 15, 2013

Related U.S. Application Data
(60) Provisional application No. 61/762,616, filed on Feb. 8, 2013.
(60) Provisional application No. 61/788,218, filed on Mar. 15, 2013.

(51) Int. Cl. C12Q 1/68

(52) U.S. Cl. CPC C12Q 1/6883
USPC 435/6.11; 435/6.12

(57) ABSTRACT

The technology described herein relates to determination of fetal lung maturity, e.g. by non-invasive methods and methods of treatment and assays relating thereto.

### FIELD OF THE INVENTION

The present invention relates to markers and methods for the assessment of fetal lung maturity, for determining the risk that a fetus will experience respiratory distress upon parturition, and for the monitoring the effects of maturity accelerating drugs on fetal lung maturity. More specifically, the present invention relates to markers of fetal lung maturity obtainable from maternal blood, and from amniotic fluid and neonatal lung aspirates.

### DESCRIPTION OF RELATED ART

Assessments of fetal lung maturity are an important tool for obstetricians and gynecologists. Decisions must often be made regarding the elective induction of labor or cesarean delivery in high risk cases, such as cases including growth-restricted fetuses, placenta previa, maternal hypertension or poorly controlled diabetic mothers. A major factor in determining whether to induce delivery is the lung maturation status of the fetus. Immature lungs are deficient in lung surfactant, a lipoprotein complex produced by Type II pneumocytes. Lung surfactant prevents the collapse of alveoli at end-expiration by reducing surface tension at the air-lipid interface. In neonates delivered with immature lungs, there is a high risk of respiratory distress syndrome caused by insufficient lung surfactant. Lung maturity assessments are therefore necessary before rational decisions regarding early delivery can be made.

When the fetus is at risk of lung immaturity and respiratory distress if delivered prematurely, treatment decisions require information about lung maturity. For example, maternal administration of corticosteroids can accelerate pneumocyte development and speed the development of surfactant secretion, but this therapy can have serious adverse effects on both fetal and maternal health. It is fore desirable for medical personnel to have access to methods of assessing fetal lung maturity which can be employed repeatedly, at frequent or serial intervals, so that the effects of prenatal treatment on lung maturity can be monitored, and the treatment can be terminated upon achieving the required effect.

In cases wherein there is no apparent likelihood of fetal lung immaturity, it is useful for gynecologists and obstetricians to have the capability of regularly and frequently monitoring the progress of fetal lung maturation. This capability would allow the construction of lung development and maturation curves. Such curves can in turn be useful in predicting likely lung abnormalities not otherwise apparent until birth, and in characterizing lung development patterns in specific populations, such as populations of pregnant mothers exposed to various risk factors.

At present, assessments of fetal lung maturity are typically made on the basis of surfactant-related biomarkers found in amniotic fluid samples. Typical assessment tests include the lecithin-to-sphingomyelin (L/S) ratio, the qualitative detection of phosphatidylglycerol (PG), and the lamellar body count (LBC) (Grenache and Gronowski, 2006). For example, immunological methods assessing fetal lung maturity by quantitation of PG in amniotic fluid are disclosed by U.S. Pat. Nos. 4,388,412 and U.S. Pat. No. 4,459,362 to Yabusaki Methods for assessing fetal lung maturity by measurement of enzymatic breakdown products of PG in amniotic fluid are disclosed in U.S. Pat. No. 5,443,989 to Alvarez and U.S. Pat. No. 5,024,936 to Macri. Methods for assessing fetal lung maturity by nonchromatographic measurements of PG in amniotic fluid are disclosed in European Patent No. EP 0230343 to Rosenthal.

Amniotic fluid based tests for assessing fetal lung maturity have significant drawbacks. Amniocentesis is an uncomfortable procedure that carries risk for both mother and fetus, and so can be employed only sporadically. Amniocentesis is contraindicated in some conditions, including anterior placental location, a low level of amniotic fluid, high risk for causing a fetal eye, umbilical cord or other structural injury, or any condition carrying a high risk of miscarriage. Because of its inherent risks, amniocentesis cannot be repeated serially in pregnancy. Amniocentesis also requires the use of specialized facilities and highly trained personnel to obtain samples of amniotic fluid.

The assessment of fetal lung maturity by means of biomarkers found in maternal circulation would remedy many of the shortcomings of amniotic fluid biomarkers, since blood samples can be obtained quickly, frequently, with little discomfort to the mother, and essentially no risk to mother or fetus. No blood-based tests of fetal lung maturity are known at present. Methods for proteomic screening of maternal serum for markers of intra-amniotic infection are disclosed in European Patent No. EP 1618388 to Rosenfeld. Methods for screening a fetus for Turner Syndrome by determining a level of the metalloprotease ADAM12 in maternal serum are disclosed in European Patent No. EP 1901074 to Wewer. None of these tests are useful for assessing fetal lung maturity. A growing body of research literature has established that nucleic acids released by fetal cells and placental trophoblasts can be recovered from maternal circulation in sufficient quantity and sufficiently intact to allow certain diagnostic tests to be performed. It is now possible, for example, to perform tests for Down, Edward, Patau syndromes and for fetal gender by analysis of circulating cell-free fetal nucleic acids (Tjoa et al., 2006, Quake et al., 2008, Akolekar et al., 2010). No maternal blood-based biomarkers of fetal lung maturity, however, exist in the prior art.

Should respiratory distress occur in a neonate after parturition, a common course of treatment is to maintain the neonate on a ventilator, and administer exogenous surfactants, until the lungs of the neonate have reached a sufficient level of maturity to allow weaning from the ventilator The weaning decision is typically made on the basis of factors other than an assessment of fetal lung maturity, centering instead on such measures as PaO2/FIO2 ratio, hemoglobin levels, and measures of cardiovascular function (Frutos-Vivar 2003). When these measures are found to be favorable, trials of spontaneous breathing are attempted, but these trials can be traumatic for distressed neonates if their lungs are still not sufficiently developed.

There remains a need for additional biomarkers and testing methods for the assessment of fetal lung maturity, especially biomarkers and methods which do not require amniotic fluid samples.

### BRIEF SUMMARY OF TILE INVENTION

The present invention provides a method for assessing fetal lung maturity by determining a level of cell free maternal serum mRNA encoding lysophospholipid acyltransferases (lysophosphotidylcholine acyltransferase 1 (LPCAT1) and/or lysophosphotidylglycerol acyltransferase 1 (LPGAT1). Both referred to synonymously herein as “LPCAT1”).

The present invention also provides a method for assessing fetal lung maturity by determining a level of LPCAT1 protein in maternal serum.

The present invention further provides a method for assessing the risk of a fetus for developing neonatal respiratory distress syndrome by determining a level of cell free maternal serum mRNA encoding LPCAT1 and comparing the level to a reference standard for risk.

The present invention still further provides a method for assessing the risk of a fetus for developing neonatal respiratory distress syndrome by determining a level of LPCAT1 protein in maternal serum and comparing the level to a reference standard for risk.

The present invention also provides a method for monitoring the effect of a treatment to accelerate lung maturation in a fetus, by determining levels of cell free maternal serum mRNA encoding LPCAT1 at a plurality of time points during treatment.

The present invention further provides a method for monitoring the effect of a treatment to accelerate lung maturation in a fetus, by determining levels of LPCAT1 protein in maternal serum mRNA at a plurality of time points during treatment.

The present invention still further provides a method for monitoring the process of fetal lung development by determining levels of cell free maternal serum mRNA encoding LPCAT1 at a plurality of time points during gestation.

The present invention also provides a method for monitoring the process of fetal lung development by determining levels of LPCAT1 protein in maternal serum at a plurality of time points during gestation.

The present invention further provides a method for assessing fetal lung maturity by determining a level of cell free amniotic fluid mRNA encoding LPCAT 1.

The present invention still further provides a method for assessing the risk of a fetus for developing neonatal respiratory distress syndrome by determining a level of cell free amniotic fluid mRNA encoding LPCAT1, and comparing the level to a reference standard for risk.

The present invention also provides methods for assessing fetal lung maturity and determining the risk of a fetus for developing neonatal respiratory distress by quantitating LPCAT1 protein in amniotic fluid.

The present invention further provides a method for assessing lung maturity in a neonate by determining a level of cell free LPCAT1 mRNA or LPCAT1 protein in a bodily fluid, the bodily fluid including neonatal lung secretions, saliva, nasal secretions, urine, and stool.

The present invention still further provides a method for determining whether the health of a neonate with respiratory distress syndrome will be jeopardized by weaning from a ventilator, by determining a level of LPCAT1 mRNA or protein in a bodily fluid, the bodily fluid including neonatal lung secretions, saliva, nasal secretions, urine, and stool.

The present invention also provides a method for assessing the lung function of any individual, including a child or adult individual, by determining a level of LPCAT1 mRNA or protein in a bodily fluid, the bodily fluid including neonatal lung secretions, saliva, nasal secretions, urine, and stool.

The present invention further provides methods for determining whether the health of any individual, including a child or adult individual, will be jeopardized by weaning from a ventilator, by determining a level of LPCAT1 mRNA or protein in a bodily fluid, the bodily fluid including lung secretions, saliva, nasal secretions, urine, and stool.

The present invention still further provides polymerase chain reaction (PCR) primers for amplifying a target sequence of human LPCAT1 cDNA and for quantitating the LPCAT1 cDNA by real-time polymerase chain reaction (RT-PCR).

The present invention also provides biomarkers for assessing fetal or neonatal lung maturity or the lung function of a child or adult individual.

The present invention also provides a method for treating a patient bearing a fetus at risk of neonatal respiratory distress syndrome by directing treatment only to a patient whose serum or amniotic fluid exhibits a level of LPCAT1 expression that is below a threshold level known to indicate sufficient lung maturity.

The present invention further provides a method for ventilation therapy wherein the weaning of an individual for a ventilator is not recommended if a level of LPCAT1 expression in a body fluid of the individual is below a threshold level known to indicate a risk that the health of the individual will be jeopardized by weaning from a ventilator.

The present invention still further provides assay kits for quantitating LPCAT1 expression in body fluids and relating the resulting LPCAT1 expression values to parameters of lung maturity and function, including a level of fetal lung maturity, a risk of neonatal respiratory distress, an effect of treatment to accelerate fetal lung development, the progress of fetal lung development, a level of fetal lung maturity in a neonate, a risk of weaning a patient from a ventilator, and a level of lung function.

### BRIEF DESCRIPTION OF THE SEVERAL VIEWS OF THE DRAWINGS

Other advantages of the present invention will be readily appreciated as the same becomes better understood by reference to the following detailed description when considered in connection with the accompanying drawings wherein:

FIG. 1A shows a graph of correlation between lamellar body counts in amniotic fluid samples and copy numbers of cell free LPCAT1 mRNA in the same amniotic fluid samples; and

FIG. 1B shows a graph of correlation between lamellar body counts in amniotic fluid samples from a population of gestating patients and copy numbers of cell free LPCAT1 mRNA in the sera of the same gestating patients.

### DETAILED DESCRIPTION OF THE INVENTION

The present invention includes novel biomarkers of fetal lung maturity and novel methods for the use of these biomarkers for the assessment of fetal lung maturity. The novel biomarkers include maternal serum-derived or amniotic fluid derived cell free mRNA species encoding lysophospholipid acyltransferases (lysophosphotidylcholine acyltransferase 1 (LPCAT1) and/or lysophosphotidylglycerol acyltransferase 1 (LPGAT1). Both referred to synonymously herein as “LPCAT1”. The novel biomarkers also include and LPCAT1 protein or fragments thereof derived from maternal serum, amniotic fluid, and neonatal lung aspirates.

It is known that cell free nucleic acids released by fetal and placental cells can be recovered from maternal circulation and analyzed to determine fetal properties such as gender and risk of Down and other syndromes (Toa et al., 2006, Quake et al., 2008, Akolekar et al, 2010). It has also been established that LPCAT1 is involved in lung surfactant production (Nakanishi et al., 2010). In a murine model, it has been shown that LPCAT1-mediated production of lung surfactants is essential for fetal transition to air breathing (Bridges et al., 2010).

The present invention includes practical applications of the discovery that LPCAT1 is an accurate indicator of human fetal lung development, and that the progress of fetal lung development can accurately and reproducibly assessed through the measurement of cell free LPCAT1 mRNA derived not only from amniotic fluid, but also from maternal blood serum.

A preferred embodiment of the present invention includes methods for assessing fetal lung maturity by measuring a level of the biomarker maternal serum fetal LPCAT1 mRNA, which as used herein refers to cell free fetal mRNA in maternal circulation, the mRNA encoding LPCAT1 or a fragment thereof. A “cell free” nucleic acid” as used herein, refers to a circulating or free-floating nucleic acid which exists outside of any intact or partially intact cell, and to a nucleic acid which exists outside of any intact or partially intact cell, but within a cellular or cell-like component such as a membrane vesicle or other membranous structure. The term “fetal lung maturity” is used herein to refer to the state of fetal lungs that are structurally and functionally prepared for postnatal respiration, especially with regard to adequate production of the protein and phospholipid lung surfactants that reduce alveolar surface tension and prevent alveolar collapse during expiration. The term “neonatal respiratory distress syndrome” is used herein to refer to the pathology produced by insufficient production of lung surfactants, and is characterized by the collapse of alveoli with each respiration, leading to muscular exhaustion, fluid buildup, diminished gas exchange, hypoxia, hypercarbia, and acidosis. The term “maternal serum” as used herein refers to any cell free blood product of a gestating female, including but not limited to serum and plasma, and to any other biological fluid that can be extracted from the tissues of a gestating female, including but not limited to lymphatic fluid, vaginal fluid, cervical fluid, saliva, urine and tears. The term “fetal mRNA” is herein used broadly, to refer to mRNA derived from either fetal tissue, placental tissues, or both. Preferably, the level of maternal serum fetal LPCAT1 mRNA is expressed in terms of copy number of LPCAT1 mRNA per cc of maternal serum, but any suitable measure of LPCAT1 mRNA can alternatively be employed. Similar methods for assessing lung maturity by measuring a level of LPCAT1 protein in maternal serum are also provided. The term “LPCAT1 protein” as used herein indicates any form of mammalian LPCAT1 protein or a fragment thereof (e.g. NCBI Gene ID: 79888). In some embodiments, a LPCAT1 protein is a protein encoded by an mRNA having the sequence of SEQ ID NO: 9 or a fragment thereof. In some embodiments, a LPCAT1 protein is a protein having the sequence of SEQ ID NO: 10 or a fragment thereof, “LPCAT1 protein” can refer to any isoform of human LPCAT1. In some embodiments, LPCAT1 protein can be the form encoded by a gene including SEQ ID NO: 8.

The assessment of fetal lung maturity through the measurement of cell free maternal serum fetal LPCAT1 mRNA or protein is by no means an obvious innovation. It is likely that most or all of the cell free fetal LPCAT1 mRNA found in circulation during the last trimester of pregnancy is not derived from fetal lung pneumocytes themselves, but from placental syncytiotrophoblasts undergoing apoptosis or necrosis under oxidative stress. In fact, levels of cell free circulating fetal DNA have been suggested to be an indicator of trophoblastic degeneration and a need for anti-oxidant treatment during pregnancy (Tjoa et al., 2006). The positive relationship between fetal lung maturation and levels of circulating cell free LPCAT1 mRNA in maternal circulation is therefore an unexpected discovery.

The assessment of fetal lung maturity through the measurement of maternal serum fetal LPCAT1 mRNA or protein represents an important advance over the prior art. The safe and noninvasive nature of blood drawing make it possible to assess fetal lung maturity in situations wherein amniocentesis cannot safely be performed. Sampling by blood draws also avoids the risk and discomfort of amniocentesis, as well as the need for costly use of specialized facilities and highly trained personnel. The present invention represents a transformative approach in obstetrics, because delivery and treatment decisions can be based on blood samples obtainable in any clinic or doctor's office. Furthermore, the capability of frequent and regular sampling makes it possible to monitor the progress of fetal lung development throughout the course of gestation. Though the use of the present invention, obstetricians are enabled to take fetal lung maturity into account when making treatment decisions regarding common obstetrical conditions, such as premature labor, premature rupture of membranes, third trimester bleeding, and fetal growth restriction. The administration of steroids to promote fetal lung maturity can also be conditioned according to actual knowledge of lung maturity.

In another embodiment of the present invention, methods are provided for assessing fetal lung maturity by measuring a level of the biomarker amniotic fetal LPCAT1 mRNA which is used herein to refer to cell free fetal mRNA in the amniotic fluid surrounding a subject fetus, the mRNA including any mRNA encoding LPCAT1 or a fragment thereof. Although lung maturity biomarkers of amniotic fluid are known, the biomarker LPCAT1 has not been employed as a biomarker in the form of cell free mRNA or in any other form. It is advantageous to add the biomarker amniotic fetal LPCAT1 mRNA to the repertoire of amniotic fluid markers of fetal lung maturity, because it can be readily and timely assayed and increases the diversity of alternative measures on which high risk treatment decisions can be made. Methods are also provided for the assessment of fetal lung maturity by measuring a level of LPCAT1 protein in amniotic fluid.

In an alternative embodiment of the present invention, methods are provided for assessing lung maturity post-natally by measuring a level of cell free LPCAT1 mRNA or LPCAT1 protein in lung aspirates of a newborn infant. These methods are useful as an objective test of lung maturity in infants experiencing respiratory distress. They are especially useful in determining when a distressed infant can be safely weaned from a ventilator. It is envisioned that other newborn bodily fluids such as saliva, nasal secretions, urine, stool and other bodily fluids may contain levels of LPCAT1 sufficient to be used to assess or correlate with lung maturity.

It is also likely that LPCAT1 levels are indicators of lung function in all individuals, including children and adult individuals. Methods are therefore also provided for assessing the lung function of a child or adult individual by measuring a level of LPCAT1 protein in lung secretions, saliva, nasal secretions, urine, and stool. Methods are also provided for assessing when an individual in respiratory distress can safely be weaned from a ventilator.

For the performance of serum-based assessment of fetal lung maturity according to a preferred embodiment of the present invention, fetal LPCAT1 mRNA is extracted from maternal blood and quantitated, preferably on the basis of copy number per cc of maternal serum. The copy number per cc value is compared to a threshold value of copy number per cc known to indicate sufficient fetal lung maturity to permit delivery with low risk of respiratory distress.

Samples can be obtained as early as 23 weeks of gestation. Preferably, LPCAT1 mRNA is quantitated by reverse transcribing the serum mRNA and performing real-time PCR (RT-PCR) on the resulting sample of cDNA. Any suitable methods of nucleic acid extraction, reverse transcription, and RT-PCR known in the art can be employed. In a preferred protocol,

Maternal samples are collected and stored on wet ice until processed by the laboratory. In no case were samples allowed to be stored in this manner for more than 12 hours before processing.

Fetal mRNA is isolated in a multi-step procedure that is similar for isolation from both maternal amniocentesis fluid or maternal serum. Cells from the collected fluids are removed by centrifugation at 1200 rpm for 15 minutes. The resulting supernatant is collected into fresh microfuge tubes. Protein is removed from the supernatants using TRizol reagent (Life technologies) using the RNA isolation procedure as detailed by the manufacturer. The resulting RNA is precipitated using isopropanol, and washed in 75% ethanol. After drying, the RNA pellet is resuspended in 20 μl RNase free water. Following isolation, the resulting RNA was subject to spectophotomety to analyze concentration and purity.

The RNA samples are immediately subject to reverse transcriptase cDNA synthesis using a CDNA synthesis kit that utilizes Superscript II reverse transcriptase and oligodT primers. Ten ul of total RNA were converted to cDNA by incubation at 42° C. for 1 h with 400 U SuperScript® II reverse transcriptase (Invitrogen), oligo(dT)24 as primer in a reaction buffer containing 50 mM Tris-HCl, pH 82, 75 mM KCl, 3 mM MgCl2, 10 mM dithiothreitol (DTT), and 02 mM dNTPs.

Pure cDNA samples were used in a Taqman type real time PCR reaction. The sequence of the forward primer for the reaction was AGGATTCTCGCAGGAAAACAG (SEQ ID NO: 1) which corresponds to base pairs 539-559 of the LPCAT1 cDNA sequence. The sequence of the reverse primer used in the reaction was TTGAAGGTAATTAGGCAGGTCC (SEQ ID NO: 2) which corresponds to complementary binding sequence of base pairs 564-586 of the LPCAT1 cDNA sequence. The sequence of the Taqman probe used in the reaction was AGAAATCAAGAGACGGGCGCAGT (SEQ ID NO: 3) which corresponds to base pairs 539-559 of the LPCAT1 cDNA sequence. The Taqman probe contained a Texas Red colormetric indicator on the 5 prime end and a Black Hole quencher on the 3 prime end. The target amplicon was a 107 bp fragment of LPCAT1 including TCAAGATCAT CAGCAATGCC TCCTGCACCA CCAACTGCTT AGCACCCCTG GCCAAGGTCA TCCATGACAA CTTTGGTATC GTGGAAGGAC TCATGACCAC AGTCCAT (SEQ ID NO: 4)

A standard real time PCR assay was used for quantitation of LPCAT copy number. The assay is as follows: cDNA 1 ul, 0.3 uM forward and reverse primer, 0.05 uM probe, TAQ DNA polymerase and corresponding buffer was used. The reaction was kept on ice until the aliqouted into ice cold PCR tubes. The tubes were immediately placed into the thermocycler and the reaction started. The reaction consisted of a hot start”, 95° C. for 150 seconds, followed by 40 cycles of amplification at 62° C. for 30 secs and 95° C. for 15 secs. The themocycler was set to read Texas Red signal.

To standardize the copy number of LPCAT in the sample a series of amplifications were conducted simultaneously with the unknown samples in which a known amount of DNA corresponding to a 125 base pair fragment of LPCAT (encompassing the sequence to be amplified) was used as target. DNA concentration corresponding to 100, 101, 102, 103 and 104 DNA copies were routinely used. From these reactions a standard curve was determined. The copy number of the unknown samples was determined from this standard curve.

As an internal control, the relative amount of cDNA added to each reaction was determined by amplification of the “housekeeping” gene glyceraldehyde 3-phosphate dehydrogenase (GAPDH). The forward primer for this reaction was AGCCTCAAGATCATCAGCAATG (SEQ ID NO: 5), corresponding to 427-448 of the GAPDH cDNA sequence. The reverse primer for the reaction was ATGGACTGTGGTCATGAGTCCTT (SEQ ID NO: 6), corresponding to the complementary binding sequence of base pairs 511-528 of the GAPDH cDNA sequence. The Taqman probe was CCAACTGCTTAGCACCCC (SEQ ID NO: 7) corresponding to base pairs 461-477 of the GAPDH cDNA sequence and it contained a 5′ Texas Red label and a 3′ Black Hole quencher. The reaction consisted of a hot start”, 95° C. for 150 seconds, followed by 40 cycles of amplification at 60μ° C. for 30 secs and 95° C. for 15 sees. The themocycler was set to read Texas Red signal.

The normalized value for LPCAT mRNA expression was calculated as the relative quantity of LPCAT divided by the relative quantity of GAPDH. Triplicate samples were run. The resulting values were multiplied by 50 to convert the mRNA expression to levels in copies/cc of starting sample accounting for dilutions during processing.

Once fetal LPCAT1 mRNA has been quantitated to obtain a value for copy number per cc maternal serum, it is next determined whether lung maturation is sufficient to permit induction of labor or cesarean section with low risk of respiratory distress. The determination is performed by comparing the copy number per cc to a known threshold copy number known to indicate sufficient lung maturity. An exemplary threshold value is 100 copies/cc. We have determined that 100 copies per cc of maternal serum or 100 copies per cc of maternal amniocentesis fluid is indicative of fetal lung maturity. It is advisable, however, for each user to calibrate the assessment test according a known reference standard biomarker of fetal lung maturity, preferably a reliable amniotic fluid biomarker. Calibration ensures that local variations in reagents and laboratory conditions do not distort the conclusions drawn from the test. One suitable reference standard biomarker is amniotic fluid LBC. Specific levels of LBC have been well established to correlate with particular levels of lung maturity (Neerhof et al., 2001).

In order to calibrate the assessment test, parallel samples of amniotic fluid and blood are drawn from each of a plurality of gestating women. Preferably, the samples are drawn at a plurality of time points over the course of gestation, beginning with samples at 23 weeks or later. Any suitable statistical correlation procedure known in the art can be used to find correlations between the LBC values of the amniotic fluid samples and LPCAT1 mRNA copy number values of the serum samples. In most cases, an LPCAT1 mRNA copy number corresponding to an LBC of 40,000 to 50,000, more preferably 50,000, will be considered the threshold for risk of neonatal respiratory distress.

If it is found that the copy number of maternal serum LPCAT1 mRNA is below the threshold for risk, treatment decisions can be at least partially on the basis of that result. If the decision at hand is whether to induce early labor, then a serum LPCAT1 mRNA copy number below the threshold value will bias the decision toward postponing delivery. A decision to commence corticosteroid treatment of the pregnant mother is also favored by a serum LPCAT1 mRNA copy number below the threshold value.

Fetal lung maturity and risk of respiratory distress can of course also be assessed through measurement of LPCAT protein in maternal serum, by methods similar to those disclosed below for the measurement of LPCAT1 protein in amniotic fluid.

Corticosteroid treatment can be executed according to any accepted protocol known in the art. An example is the protocol recommended by the American College of Obstetricians and Gynecologists (AGOG, 2011).

A maternal serum LPCAT1 mRNA value below the threshold for risk can also indicate the ation of gene therapy of the fetus to reduce the risk of respiratory distress after parturition. Any gene therapy known an the art to cause a sufficient increase in fetal lung surfactant, over a sufficient span of time to reduce the risk of neonatal respiratory distress, can be employed. For example, as it is known that LPCAT1 is a critical enzyme in the biosynthetic pathway of surfactant production of lung pneumocytes, the gene therapy can be targeted towards increasing LPCAT1 expression in fetal pneumocytes of other cells of the fetal lung. The expression vectors developed by Harayama et al. (2009) can for example be readily adapted to therapeutic purposes. Even transiently increased expression of LPCAT1 can be sufficient reduce the risk of respiratory distress in the hours and days after parturition.

If treatments to accelerate fetal lung development are administered, it is important to monitor their effects. Corticosteroids, with their serious side effects, particularly in diabetics, should not be given if the LPCAT1 level already determines that the fetal lungs are mature, or continued if the treatment is not producing the desired effect. If a treatment is failing, it is essential that it be discontinued and a different treatment substituted if possible. The present invention provides monitoring tests for determining the effect of a treatment to accelerate lung maturation in a fetus, by measuring a copy number value of serum fetal LPCAT1 mRNA per unit volume of maternal serum at each of a plurality of successive time points over the course of the treatment. This method represents a capability heretofore unavailable in the field of obstetrics. Amniotic fluid biomarkers of fetal lung development are not suited for monitoring therapy, because even in cases wherein amniocentesis is not contraindicated, the procedure cannot be performed at the daily or even hourly intervals likely to be required for monitoring. In contrast, maternal serum levels of LPCAT1 mRNA can be determined as frequently as blood draws can be made.

The effects of a treatment to accelerate fetal lung maturation can of course also be assessed through measurement of LPCAT1 protein in maternal serum, by methods similar to those disclosed below for the measurement of LPCAT1 protein in amniotic fluid.

To monitor the effects of a treatment to accelerate lung maturation, a sample of maternal serum is collected at each of a plurality successive time points, the first time point being either prior to or after the initiation of the treatment. A value for the copy number of serum fetal LPCAT1 mRNA per unit volume of maternal serum is determined at each of the successive time points, and the difference is calculated between the value at each time point and the value at the immediately preceding time point. If the treatment is accelerating fetal lung development, this will immediately be apparent from increasing values of copy number of serum fetal LPCAT1 mRNA per unit volume. Decisions regarding further treatment, such as continuing, discontinuing, or changing treatment can be based at least in part on the results of the successive assessments.

The capability of the present invention to provide frequent and repeated assessments of fetal lung maturity can of course by utilized by one skilled in the art for purposes other than the monitoring of a treatment. The invention is also useful for tracking the development of fetal lungs for any purpose.

In another embodiment, the present invention includes methods for assessing fetal lung maturity basis of levels of LPCAT1 in amniotic fluid. LPCAT1 levels can be measured either as a copy number of cell free amniotic fluid fetal LPCAT1 mRNA, or as level of LPCAT1 protein in amniotic fluid. To assess a level of lung maturity, the level of cell free LPCAT1 mRNA or protein is determined and compared to a reference standard relating levels of amniotic LPCAT1 mRNA or protein to levels of fetal lung maturity. A level of lung maturity is then assessed by determining the value of the reference standard that corresponds to the level of LPCAT1 mRNA or protein found in the amniotic fluid sample. The level of amniotic LPCAT1 mRNA or protein can be used to determine whether lung maturation is sufficient to permit induction of labor with low risk of respiratory distress. As previously described for serum LPCAT1 mRNA, the determination is performed by comparing the observed level of LPCAT1 mRNA or protein to a level of LPCAT1 mRNA or protein known to indicate sufficient lung maturity. Also as previously discussed, the assessment test is preferably calibrated by a user against a suitable reference marker, preferably amniotic LBC. In most cases, a LPCAT1 mRNA copy number corresponding to an LBC of 40,000 to 50,000, more preferably 50,000 will be considered the threshold for risk of neonatal respiratory distress.

Where cell free amniotic fluid fetal LPCAT1 mRNA is to be determined, the methods previously disclosed for quantitating maternal serum cell free fetal LPCAT1 mRNA can be applied, with modifications made as required for the different physical and chemical environment of amniotic fluid. Where LPCAT1 protein is to be determined, immunological assays against any suitable LPCAT1 protein can be utilized, including but not limited to noncompetitive sandwich assays, such as enzyme linked immunosorbent assays (ELISA), competitive inhibition assays such as enzyme immunoassays (EIA), radioimmunoassay (RIA), and fluorescence polarization immunoassays (FPIA). Other assays such as quantitative western blotting, and fluorescent bead based immunoassays of any suitable type known in the art, are also within the scope of the present invention.

In general, sandwich assays are the preferred for their high sensitivity, and are well known to those skilled in the art. Both polyclonal and monoclonal antibodies can be used in the assays. These immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, Molecular Cloning: A Laboratory Manual, Cold Springs Harbor, New York, 1989. Standard methods in immunology known in the art and not specifically described are generally followed as in Stites et al, (eds), Basic and Clinical Immunology (8th Edition), Appleton & Lange, Norwalk, Conn. (1994) and Mishell and Shiigi (eds), Selected Methods in Cellular Immunology. Freeman and Co., New York (1980).

Alternatively, LPCAT1 protein can be determined in terms of lysophosphatidylcholine acyltransferase 1 activity by any suitable enzymatic assay. An exemplary assay is the lysophosphatidylcholine acyltransferase 1 activity assay disclosed by Harayama et al. (2009). Alternative assays are readily adapted from the protocols found in standard enzyme handbooks such as Eisenthal R and Danson R J, eds, Enzyme assays: a practical approach (1992).

The present invention also provides a test to determine a risk that the health of a neonate suffering from respiratory distress syndrome, and who has been placed on a ventilator, will be jeopardized by weaning from the ventilator. The test is based on a level of LPCAT1 mRNA or protein in the respiratory secretions of the neonate. Information provided by the test will greatly increase the accuracy of decisions regarding whether to wean a neonate in respiratory distress from a ventilator. Criteria for weaning currently in use are based on blood gas levels and hemodynamic factors that provide no direct measure of the actual state of lung maturity. The present invention provides this information, and so enables physicians to make more rational decisions about weaning.

In the preferred embodiment of the neonatal lung maturity test of the present invention, a sample of respiratory secretions of a neonate is collected by aspiration. A level of LPCAT1 per cc of secretions is determined. As previously described, this level can be determined in terms of either cell free LPCAT1 mRNA or of LPCAT1 protein, by methods previously described. The level of LPCAT1 is then compared to a reference standard relating a level of LPCAT1 to a threshold below which the lung maturity of a neonate is insufficient to support independent respiration, to the extent that the health of the neonate will be jeopardized by weaning from the ventilator. The resulting conclusion can be used as a factor in a decision as to whether or not to wean the neonate from the ventilator, give artificial surfactant or other therapies to support respiration.

The method for assessing neonatal lung maturity and the risk of weaning from a ventilator need not be confined to lung aspirates. The assessment may also be performed with samples of other bodily fluids, including saliva, nasal secretions, urine, and stool.

LPCAT1 can also be utilized as a biomarker in methods for assessing lung function in children and adults, and for assessing the risk of weaning children or adults from a ventilator. The previously described methods for measuring LPCAT1 mRNA or protein in maternal serum or amniotic fluid are readily adapted for assessments in lung aspirates and in other bodily fluids, including saliva, nasal secretions, urine, and stool.

Also within the scope of the sent invention are improved methods of treatment for conditions related to lung immaturity and to lung function in general. These methods permit practitioners to target treatments more specifically to individuals who are in need of treatment, and to avoid treating individuals not in need of treatment, and who would needlessly experience the adverse side effects of treatment. Specifically, the present invention includes a method of treating a patient bearing a fetus at risk of neonatal respiratory distress syndrome, including the steps of determining that a level of LPCAT1 expression in a sample of a body fluid is below a threshold level known to indicate a risk of respiratory distress syndrome, and administering treatment only to patients bearing fetuses indicated to be at risk. The treatments can include corticosteroid treatment to accelerate lung maturation, gene therapy to increase the expression of LPCAT1 protein expression in fetal lung tissue, or recommending against preterm delivery. The body fluids can include maternal serum and amniotic fluid. The level of LPCAT1 expression can be defined as a level of cell free LPCAT1 mRNA or of LPCAT1 protein per cc of a body fluid.

The present invention also includes improved methods for ventilator therapy. Specifically, the invention includes methods for determining whether a level of LPCAT1 expression in a sample of a body fluid of the individual is above a below a level known to indicate a risk that the health of the individual will be jeopardized by weaning from a ventilator, and recommending against the weaning of individuals exhibiting a level of LPCAT1 expression that is lower than the threshold level. The invention allows a practitioner to more rationally target the weaning process to those individuals whose health is not likely to be threatened by removal from a ventilator, and this targeting capability increases the overall effectiveness of ventilation therapy. LPCAT1 levels can be measured in body fluids including, but not limited to lung secretions, saliva, nasal secretions, urine, and stool by techniques described previously.

The present invention also includes assays for determining the concentration of cell free LPCAT1 mRNA or LPCAT1 protein in body fluids. The assays are provided as kits including reagents for measuring LPCAT1 mRNA or protein, and instructions for relating the results of the assays to parameters of lung maturity and lung function. The instructions can include standard curves or standard tables relating specific concentrations of LPCAT1 mRNA or protein to particular levels of lung function or risk of respiratory distress. For example, a kit for determining a level of risk of a fetus for developing neonatal respiratory distress syndrome can include an instruction that cell free LPCAT1 mRNA lower than a threshold concentration of 100 copies per cc of maternal serum indicates a risk of neonatal respiratory distress syndrome.

In general, the kits for quantitation of cell free LPCAT1 mRNA include at least one nucleic acid reagent specifically hybridizable to at least one target nucleic acid sequence encoding LPCAT1; and a quantitation system for quantitating the hybridization of the nucleic acid reagent with the target sequence, and for deriving a concentration value of LPCAT1 mRNA per unit volume of the sample of body fluid. The preferred quantitation system provided in the assay kit is a PCR system wherein the target nucleic acid sequence encoding LPCAT1 is a cDNA transcribed from said cell free LPCAT1 mRNA, and the nucleic acid reagent is an oligonucleotide primer pair for amplifying said LPCAT1 cDNA, preferably including SEQ ID NO:1 and SEQ ID NO:2, which amplify including SEQ ID NO: 4, The preferred assay kit includes reagents for determining a concentration value of LPCAT1 mRNA per unit volume of a sample of body fluid by real time PCR (RT-PCR). Optionally, the kit includes additional reagents for the standardization of the RT-PCR results, such as a primer pair for a housekeeping gene, a reporter probe, and buffers, washes, and diluents as required. Exemplary reagents are described in Example 1.

Specific assay kits within the scope of the present invention include an assay kit for the antenatal assessment of fetal lung maturity, which can be used to quantitate cell free LPCAT1 mRNA in samples of maternal serum or amniotic fluid, and which includes instructions for relating the results to a level of fetal lung maturity; an assay kit for assessing a risk that a fetus will develop neonatal respiratory distress syndrome, which can be used to quantitate cell free LPCAT1 mRNA in samples of maternal serum or amniotic fluid, and which includes instructions for relating the results to a risk of neonatal respiratory distress syndrome.

Also included in the present invention are three assay kits that can be used to quantitate cell free LPCAT1 mRNA in samples derived from neonatal and postnatal individuals, the samples including, but not limited to, lung secretions, saliva, nasal secretions, urine, and stool. One of these assay kits is an assay kit for the assessment of lung maturity in a neonate, and it includes instructions for relating the results to a level of lung maturity. Another of these assay kits is an assay kit for the assessment of risk that the health of the individual will be jeopardized by weaning from a ventilator, and it includes instructions for relating the results to a risk of weaning from a ventilator. A third assay kit is an assay kit for the assessment of lung function in any individual, and it includes instructions for relating the results to a level of lung function.

Also included in the present invention are two assay kits that can be used to quantitate cell-free LPCAT1 mRNA in a plurality of sequential samples of maternal serum or amniotic fluid. One of these assay kits is an assay kit for monitoring the progress of lung maturation in a fetus, and it includes instructions for relating differences in cell free LPCAT1 mRNA concentrations between sequential samples to the progress of fetal lung maturation. The other assay kit is an assay kit for determining the effect of a treatment to accelerate fetal lung development, and it includes instructions for relating differences in cell free LPCAT1 mRNA concentrations between sequential samples to an effect of the treatment on fetal lung maturity.

Also within the scope of the present invention is any assay kit which includes reagents for quantitating cell free LPCAT1 mRNA by directly or indirectly quantitating the hybridization of a nucleic acid reagent with nucleic acid sequence encoding LPCAT1, and which includes instructions for relating the results to a level of lung maturity or function. Examples include, but are not limited to, northern blotting, slot blotting, dot hybridization, and DNA microarrays. These kits are readily assembled according to protocols such as those found in Ausubel et al. (eds) Current protocols in molecular biology, John Wiley & Sons, New York, N.Y., 1994-2013.

Also within the scope of the present invention are immunoassay kits for determining the concentration of LPCAT1 protein in body fluids. The immunoassay kits preferably include an immunosensor for quantitating an immunoreaction between at least one antibody and LPCAT1 protein, and for providing a concentration value of LPCAT1 protein per unit volume of the sample of body fluid. The preferred immunoassay kits also provide instructions for relating a concentration value of LPCAT1 protein per unit volume of the sample of body fluid to the previously described parameters of lung maturity and lung function.

The preferred immunoassay kit includes an immunosensor including a sandwich immunoassay. This type of assay includes a capture antibody specifically reactive with a first epitope or LPCAT1 and a detection antibody specifically reactive with a second epitope of LPCAT1.

Preferably, a solid support is included to bind and immobilize the capture antibody. The detection antibody is preferably conjugated or bound to a signal-generating label such as an enzyme. Upon reaction with a sample of a body fluid, LPCAT1 protein becomes “sandwiched” between the capture antibody and the detection antibody. The kit optionally includes diluents, blockers, and washes for use in washing away unbound sample and any non-specifically bound reagents. The amount of detection antibody remaining on the solid support is measured by the signal produced by the label, which is proportional to the amount of LPCAT1 protein in the sample.

Suitable pairs of capture and detection antibodies for the assay of LPCAT1 protein are readily found in sources such as Abnova.com (www.abnova.com/products/products_list.asp?classid=AIAE00000000&OwnClass=2&gclid=CKX-i83997UCFYpDMgod51AAnQ). The solid support included in a kit can be, but is not limited to, a plate, e.g. a polystyrene, nylon, or glass plate, a membrane, e.g. a nitrocellulose, porous synthetic polymer or glass fiber membrane, beads, latex particles, magnetic particles, and the like. Detection labels included in the kit can include enzymes such as peroxidase, and alkaline phosphatase, a chemiluminscent system; a fluorescent dye such as fluorescein or Texas Red; gold colloid; colored latex; and an isotope.

Specific sandwich assays within the scope of the invention include an assay kit for the antenatal assessment of fetal lung, maturity, which can be used to quantitate LPCAT1 protein in samples of maternal serum or amniotic fluid, and which includes instructions for relating the results to a level of fetal lung maturity; and an assay kit for assessing a risk that a fetus will develop neonatal respiratory distress syndrome, which can be used to quantitate LPCAT1 protein samples of maternal serum or amniotic fluid, and which includes instructions for relating the results to a risk of neonatal respiratory distress syndrome.

Also included in the present invention are three assay kits that can be used to quantitate LPCAT1 protein in samples derived from neonatal and postnatal individuals, the samples including, but not limited to, lung secretions, saliva, nasal secretions, urine, and stool. One of these assay kits is an assay kit for the assessment of lung maturity in a neonate, and it includes instructions for relating the results to a level of lung maturity. Another of these assay kits is an assay kit for the assessment of risk that the health of the individual will be jeopardized by weaning from a ventilator, and it includes instructions for relating the results to a risk of weaning from a ventilator. A third assay kit is an assay kit for the assessment of lung function in any individual, and it includes instructions for relating the results to a level of lung function.

Also included in the present invention are two assay kits that can be used to quantitate LPCAT1 protein in a plurality of sequential samples of maternal serum or amniotic fluid. One of these assay kits is an assay kit for monitoring the progress of lung maturation in a fetus, and it includes instructions for relating differences in LPCAT1 protein concentrations between sequential samples to the progress of fetal lung maturation. The other assay kit is an assay kit for determining the effect of a treatment to accelerate fetal lung development, and it includes instructions for relating differences in cell free LPCAT1 mRNA concentrations between sequential samples to an effect of the treatment on fetal lung maturity.

The foregoing disclosed compositions and methods focus primarily on assessing lung maturity in the human fetus and neonate through the use of biomarkers including LPCAT1, but they are readily adapted for assessing lung maturity in the fetus of any mammalian species.

The invention is further described in detail by reference to the following experimental examples. These examples are provided for the purpose of illustration only, and are not intended to be limiting unless otherwise specified. Thus, the present invention should in no way be construed as being limited to the following examples, but rather, be construed to encompass any and all variations which become evident as a result of the teaching provided herein.

Some embodiments of the technology described herein can be defined according to any of the following numbered paragraphs:

1. A method for antenatally assessing fetal lung maturity, including the steps of:
collecting a sample of maternal serum;
obtaining cell free messenger RNA (mRNA) from the sample of maternal serum;
determining a level of cell free lysophospholipid acyltransferase 1 (LPCAT1) mRNA in the sample of maternal serum;
comparing the level of cell free LPCAT1 mRNA in the sample of maternal serum to a reference standard relating a level of cell free LPCAT1 mRNA to a level of fetal lung maturity;
determining the level of lung maturity of a fetus corresponding to the level of cell free LPCAT1 mRNA in the sample; and assessing the level of lung maturity of a fetus.
2. The method of paragraph 1, wherein the amount of cell free LPCAT1 mRNA in the sample of maternal serum is further defined as a copy number of cell free LPCAT1 mRNA per cc of amniotic fluid.
3. The method of paragraph 2, further including, after the step of obtaining cell free mRNA, the step of reverse transcribing the mRNA and quantitating by reverse transcriptase polymerase chain reaction (RT-PCR) the resulting LPCAT1 cDNA amplicon.
4. The method of paragraph 3, wherein the quantitating step further includes the steps of amplifying the LPCAT1 cDNA with a forward primer including an oligonucleotide with at least 95% identity with SEQ ID NO: 1 or the exact complement thereof, and with a reverse primer including a including an oligonucleotide with at least 95% identity with SEQ ID NO: 2 or the exact complement thereof, and quantitating a PCR product including SEQ ID NO: 4 with a fluorescent reporter probe including an oligonucleotide including SEQ ID NO: 3 or the exact complement thereof.
5. A method for assessing the risk of a fetus for developing neonatal respiratory distress syndrome, including the steps of:

collecting a sample of maternal serum;

obtaining cell free messenger RNA (mRNA) from the sample of maternal serum;

determining a level of cell free lysophospholipid acyltransferase 1 (LPCAT1) mRNA in the sample of maternal serum;

comparing the level of cell free LPCAT1 mRNA in the sample of maternal serum to a threshold level of cell free LPCAT1 mRNA, wherein a level of cell free LPCAT1 mRNA below the threshold level indicates risk that a fetus will develop neonatal respiratory distress syndrome; and

determining whether risk a fetus is at risk of neonatal respiratory distress syndrome.

6. The method of paragraph 5 wherein the level of cell free LPCAT1mRNA in the sample of maternal serum is further defined as a copy number of cell free LPCAT1mRNA per cc of maternal serum.
7. The method of paragraph 6, wherein the threshold level of cell free LPCAT1mRNA is further defined as 100 copies of cell free LPCAT1mRNA per cc of maternal serum.
8. The method of paragraph 6, wherein the threshold level of cell free LPCAT1mRNA is further defined as a level of cell free LPCAT1 mRNA known to be correlated to a level of an amniotic fluid biomarker associated with fetal risk for developing neonatal respiratory distress syndrome.
9. The method of paragraph 8, wherein the amniotic fluid biomarker is further defined as lamellar body count (LBC), and the threshold level of cell free LPCAT1mRNA is further defined as a level of cell free LPCAT1 mRNA known to be correlated with an amniotic fluid LBC of 40,000-50,000 lamellar bodies/μl amniotic fluid.
10. The method of paragraph 9, wherein the threshold level of cell free LPCAT1 mRNA is further defined as a level of cell free LPCAT1 mRNA known to be correlated with an amniotic fluid LBC of 50,000 lamellar bodies/μl amniotic fluid.
11. The method of paragraph 5, further including, after the step of obtaining cell free mRNA, the step of reverse transcribing the mRNA and quantitating by reverse transcriptase polymerase chain reaction (RT-PCR) the resulting LPCAT1 cDNA amplicon.
12. The method of paragraph 11, wherein the quantitating step further includes the steps of amplifying the LPCAT1 cDNA with a forward primer including an oligonucleotide with at least 95% identity with SEQ ID NO: 1 or the exact complement thereof, and with a reverse primer including a including an oligonucleotide with at least 95% identity with SEQ ID NO: 2 or the exact complement thereof, and quantitating a PCR product including SEQ ID NO: 4 with a fluorescent reporter probe including an oligonucleotide including SEQ ID NO: 3 or the exact complement thereof.
13. The method of paragraph 5, additionally including the steps of:

determining that the copy number of serum fetal LPCAT1 per unit volume of maternal serum is below the threshold level known to indicate sufficient lung maturity; and treating the pregnant mother or the fetus to reduce the likelihood of respiratory distress at parturition.

14. The method of paragraph 13, wherein the step of treating a pregnant mother or a fetus is further defined as administering corticosteroid treatments to the pregnant mother to accelerate lung maturation.
15. The method of paragraph 13, wherein the step of treating the pregnant mother or the fetus is further defined as recommending against preterm delivery.
16. The method of paragraph 13, wherein the step of treating a pregnant mother or a fetus is further defined as administering gene therapy to the fetus.
17. The method of paragraph 16, wherein the step of administering gene therapy further includes the steps of administering to a fetus a sufficient quantity of an expression vector encoding LPCAT1 and inducing in the lung tissue of a fetus a sufficient expression of LPCAT1 to prevent respiratory distress upon parturition.
18. A method for determining the effect of a treatment to accelerate lung maturation in a fetus, including the steps of:

collecting a sample of maternal serum at each of a plurality successive time points, the first time point being either prior to or after the initiation of the treatment;

obtaining cell free messenger RNA (mRNA) from the sample of maternal serum,

determining a copy number value of serum fetal lysophospholipid acyltransferase 1 (LPCAT1) mRNA per unit volume of maternal serum at each of the successive time points over the course of the treatment, the copy number value of serum LPCAT1 mRNA per unit volume of maternal serum indicating a level of lung maturation;

comparing the copy number value of serum fetal LPCAT1 mRNA per unit volume of maternal serum at each time point to the copy number value of serum fetal LPCAT1 mRNA per unit volume of maternal serum at its immediately preceding time point over the course of the treatment;

calculating the differences among copy number values of serum fetal LPCAT1 mRNA per unit over the successive time points over the course of the treatment; and

determining the effect of a treatment to accelerate lung maturation in a fetus, with increasing copy number values of serum fetal LPCAT1 mRNA per unit volume of maternal serum over the course of the treatment indicating the acceleration of fetal lung maturation.

19. The method of paragraph 18, further including the step of making a treatment recommendation based on the step of monitoring the effect of monitoring the effect of the treatment to accelerate lung maturation in a fetus, the recommendation selected from the group including continuation of the treatment, discontinuation of the treatment, and initiation of a different treatment.
20. The method of paragraph 18, further including, after the step of obtaining cell free mRNA, the step of reverse transcribing the mRNA and quantitating by reverse transcriptase polymerase chain reaction (RT-PCR) the resulting LPCAT1 cDNA amplicon.
21. The method of paragraph 20, wherein the quantitating step further includes the steps of amplifying the LPCAT1 cDNA with a forward primer including an oligonucleotide with at least 95% identity with SEQ ID NO: 1 or the exact complement thereof, and with a reverse primer including a including an oligonucleotide with at least 95% identity with SEQ ID NO: 2 or the exact complement thereof, and quantitating a PCR product including SEQ ID NO: 4 with a fluorescent reporter probe including an oligonucleotide including SEQ ID NO: 3 or the exact complement thereof.
22. A method for monitoring the progress of fetal lung development, including the steps of:

collecting a sample of maternal serum at each of a plurality successive time points during gestation;

determining a copy number value of serum fetal lysophospholipid acyltransferase 1 (LPCAT1) mRNA per unit volume of maternal serum at each of the successive time points over the course of gestation, the copy number value of serum fetal LPCAT1 mRNA per unit volume of maternal serum indicating a level of lung maturation;

comparing the copy number value of serum fetal LPCAT1 mRNA per unit volume of maternal serum at each time point to the copy number value of serum fetal LPCAT1 mRNA per unit volume of maternal serum at its immediately preceding time point over the course of gestation;

calculating differences among copy number values of serum fetal LPCAT1 mRNA per unit over the successive points over the course of gestation, wherein the differences represent the progress of fetal lung development; and

monitoring the progress of fetal lung development.

23. A method for antenatally assessing fetal lung maturity, including the steps of:

collecting a sample of amniotic fluid;

obtaining cell free messenger RNA (mRNA) from the sample of amniotic fluid;

determining a level of cell free lysophospholipid acyltransferase 1 (LPCAT1) mRNA in the sample of amniotic fluid;

comparing the level of cell free LPCAT1 mRNA in the sample of amniotic fluid to a reference standard relating a level of cell free amniotic LPCAT1 mRNA to a level of fetal lung maturity;

determining the level of lung maturity of a fetus corresponding to the level of cell free LPCAT1 mRNA in the amniotic fluid sample of maternal serum according to the reference standard; and

assessing the level of lung maturity of a fetus.

24. The method of paragraph 23, wherein the level of cell free LPCAT1 mRNA in the sample of maternal serum is further defined as a copy number of cell free LPCAT1 mRNA per cc of amniotic fluid.
25. The method of paragraph 24, further including, after the step of obtaining cell free mRNA, the step of reverse transcribing the mRNA and quantitating by reverse transcriptase polymerase chain reaction (RT-PCR) the resulting LPCAT1 cDNA amplicon.
26. The method of paragraph 25, wherein the quantitating step further includes the steps of amplifying the LPCAT1 cDNA with a forward primer including an oligonucleotide with at least 95% identity with SEQ ID NO: 1 or the exact complement thereof, and with a reverse primer including a including an oligonucleotide with at least 95% identity with SEQ ID NO: 2 or the exact complement thereof, and quantitating the a PCR product including SEQ ID NO: 4 with a fluorescent reporter probe including an oligonucleotide including SEQ ID NO: 3 or the exact complement thereof.
27. A method for assessing the risk of a fetus for developing neonatal respiratory distress syndrome, including the steps of:

collecting a sample of amniotic fluid;

obtaining cell free messenger RNA (mRNA) from the sample of amniotic fluid;

determining a level of cell free lysophospholipid acyltransferase 1 (LPCAT1) mRNA in the sample of amniotic fluid;

comparing the level of cell free LPCAT1 mRNA in the sample of amniotic fluid to a threshold level of cell free LPCAT1 mRNA, wherein a level of cell free LPCAT1 mRNA below the threshold level indicates risk that a fetus will develop neonatal respiratory distress syndrome; and

determining whether a fetus is at risk of neonatal respiratory distress syndrome.

28. The method of paragraph 27 wherein the level of cell free LPCAT1mRNA in the sample of amniotic fluid is further defined as a copy number of cell free LPCAT1mRNA per cc of amniotic fluid.
29. The method of paragraph 28, wherein the threshold level of cell free LPCAT1mRNA is further defined as 100 copies of cell free LPCAT1mRNA per cc of maternal serum.
30. The method of paragraph 27, wherein the threshold level of cell free amniotic fluid LPCAT1mRNA is further defined as a level of cell free LPCAT1 mRNA known to be correlated to a level of a second amniotic fluid biomarker associated with fetal risk for developing neonatal respiratory distress syndrome.
31. The method of paragraph 30, wherein the second amniotic fluid biomarker is further defined as lamellar body count (LBC), and the threshold level of cell free amniotic LPCAT1mRNA is further defined as a level of cell free LPCAT1 mRNA known to be correlated with an amniotic fluid LBC of 40,000-50,000 lamellar bodies/μl amniotic fluid.
32. The method of paragraph 30, wherein the threshold level of cell free amniotic fluid LPCAT1mRNA is further defined as a level of cell free amniotic fluid LPCAT1 mRNA known to be correlated with an amniotic fluid LBC of 40,000 lamellar bodies/μl amniotic fluid.
33. The method of paragraph 27, further including, after the step of obtaining cell free mRNA, the step of reverse transcribing the mRNA and quantitating by reverse transcriptase polymerase chain reaction (RT-PCR) the resulting LPCAT1 cDNA amplicon.
34. The method of paragraph 33, wherein the quantitating step further includes the steps of amplifying the LPCAT1 cDNA with a forward primer including an oligonucleotide with at least 95% identity with SEQ ID NO: 1 or the exact complement thereof, and with a reverse primer including a including an oligonucleotide with at least 95% identity with SEQ ID NO: 2 or the exact complement thereof, and quantitating a PCR product including SEQ ID NO: 4 with a fluorescent reporter probe including an oligonucleotide including SEQ ID NO: 3 or the exact complement thereof.
35. The method of paragraph 27, additionally including the steps of:

determining that the copy number of serum fetal LPCAT1 per cc of amniotic fluid is below the threshold level of known to indicate sufficient lung maturity; and

treating the pregnant mother or the fetus to reduce the likelihood of respiratory distress at parturition.

36. The method of paragraph 35, wherein the step of treating a pregnant mother or a fetus is further defined as administering corticosteroid treatments to the pregnant mother to accelerate lung maturation.
37. The method of paragraph 35, wherein the step of treating the pregnant mother or the fetus is further defined as recommending against preterm delivery.
38. The method of paragraph 35, wherein the step of treating a pregnant mother or a fetus is further defined as administering gene therapy to the fetus.
39. The method of paragraph 38, wherein the step of administering gene therapy further includes the steps of administering to a fetus a sufficient quantity of an expression vector encoding LPCAT1 and inducing in the lung tissue of a fetus a sufficient expression of LPCAT1 to prevent respiratory distress upon parturition.
40. A method for antenatally assessing fetal lung maturity, including the steps of:

collecting a sample of amniotic fluid;

measuring a level of lysophospholipid acyltransferase 1 (LPCAT1) protein in the sample of amniotic fluid;

comparing the level of LPCAT1 protein in the sample of amniotic fluid to a reference standard relating a level of LCAT1 protein to a level of fetal lung maturity;

determining the level of lung maturity of a fetus corresponding to the LPCAT1 protein in the sample of amniotic fluid according to the reference standard; and

assessing the level of lung maturity of a fetus.

41. The method of paragraph 40, wherein the measuring step is further defined as measuring a level of LPCAT1 protein by means of an immunological assay with an anti-LPCAT1 antibody.
42. The method of paragraph 41, wherein the immunological assay is selected from the group including an ELISA, a western blot, and a fluorescent bead based immunoassay.
43. The method of paragraph 41 wherein the level LPCAT1 protein in the sample of amniotic fluid is further defined as a weight LPCAT1 protein per cc of amniotic fluid.
44. The method of paragraph 40, wherein the measuring step is further defined as measuring a level of LPCAT1 protein by means of an assay of lysophospholipid acyltransferase activity.
45. The method of paragraph 44, wherein the level of LPCAT1 protein is further defined as a measure selected from the group consisting of a weight LPCAT1 protein per cc of amniotic fluid and a number of units of lysophospholipid acyltransferase activity per cc amniotic fluid.
46. A method for assessing the risk of a fetus for developing neonatal respiratory distress syndrome, including the steps of:

collecting a sample of amniotic fluid;

measuring a level of lysophospholipid acyltransferase 1 (LPCAT1) protein in the sample of amniotic fluid;

comparing the level of LPCAT1 protein in the sample of amniotic fluid to a threshold level of LPCAT1 protein, wherein a level of LPCAT1 protein below the threshold level indicates risk that a fetus will develop neonatal respiratory distress syndrome; and

determining whether risk a fetus is at risk of neonatal respiratory distress syndrome.

47. The method of paragraph 46, wherein the measuring step is further defined as measuring a level of LPCAT1 protein by means of an immunological assay with an anti-LPCAT1 antibody.
48. The method of paragraph 47, wherein the immunological assay is selected from the group including an ELISA, a western blot, and a fluorescent bead based immunoassay.
49. The method of paragraph 47 wherein the level LPCAT1 protein in the sample of amniotic fluid is further defined as a weight LPCAT1 protein per cc of amniotic fluid.
50. The method of paragraph 46, wherein the measuring step is further defined as measuring a level of LPCAT1 protein by means of an assay lysophospholipid acyltransferase activity.
51. The method of paragraph 50, wherein the level of LPCAT1 protein is further defined as a measure selected from the group consisting of a weight LPCAT1 protein per cc of amniotic fluid and a number of units of lysophospholipid acyltransferase activity per cc amniotic fluid.
52. The method of paragraph 46, wherein the threshold level of LPCAT1 protein is further defined as a level of LPCAT1 protein known to be correlated to a level of a second amniotic fluid biomarker associated with fetal risk for developing neonatal respiratory distress syndrome.
53. The method of paragraph 52, wherein the second amniotic fluid biomarker is further defined as lamellar body count (LBC), and the threshold level of LPCAT1 protein is further defined as a level of LPCAT1 protein known to be correlated with an amniotic LBC of 40,000-50,000 lamellar bodies/μl amniotic fluid.
54. The method of paragraph 53, wherein the threshold level of LPCAT1 protein is further defined as a level of LPCAT1 protein known to be correlated with an amniotic LBC of 40,000 lamellar bodies/μl amniotic fluid.
55. The method of paragraph 46, additionally including the steps of:

Determining that the level of LPCAT1 protein in the sample of amniotic fluid is below the threshold level of known to indicate sufficient lung maturity; and

Treating the fetus to reduce the likelihood of respiratory distress at parturition.

56. The method of paragraph 55, wherein the step of treating the fetus is further defined as administering corticosteroid treatments to its mother to accelerate lung maturation.
57. The method of paragraph 55, wherein the step of treating, the fetus is further defined as recommending against preterm delivery.
58. The method of paragraph 55 wherein the step of treating the fetus is further defined as administering gene therapy to the fetus.
59. The method of paragraph 50, wherein the step of administering gene therapy further includes the steps of administering to a fetus a sufficient quantity of an expression vector encoding LPCAT1 and inducing in the lung tissue of a fetus a sufficient expression of LPCAT1 to prevent respiratory distress upon parturition.
60. A method for assessing lung maturity in a neonate, including the steps of:

collecting a sample of a bodily fluid from a neonate;

determining a level of lysophospholipid acyltransferase 1 (LPCAT1) in the sample of bodily fluid;

comparing the level of LPCAT1 in the sample of a bodily fluid to a reference standard relating a level of LPCAT1 to a level of neonatal lung maturity;

determining the level of lung maturity of a neonate corresponding to the level of LPCAT1 in the sample of bodily fluid; and

assessing the level of lung maturity of a neonate,

wherein the sample of a bodily fluid is further defined as a sample of a fluid selected from the group consisting of lung secretions, saliva, nasal secretions, urine, and stool.

61. The method of paragraph 60, wherein the level of LPCAT1 in the sample of a bodily fluid is further defined as a copy number of cell free LPCAT1 mRNA per unit volume of bodily fluid, as determined according to the method of paragraph 1.
62. The method of paragraph 60, wherein level of LPCAT1 in the sample of a bodily fluid is further defined as a level of LPCAT1 protein in the sample of a bodily fluid, as determined according to the method of paragraph 40.
63. A method for assessing the risk that the health of a neonate with respiratory distress syndrome will be jeopardized by weaning from a ventilator, including the steps of:

collecting a sample of a bodily fluid from a neonate;

determining a level of lysophospholipid acyltransferase 1 (LPCAT1) in the sample of a bodily fluid;

Comparing the level of LPCAT1 in the sample of respiratory secretions to a threshold level of LPCAT1, wherein a level of LPCAT1 below the threshold level indicates risk that the lung maturity of a neonate is insufficient to support independent respiration; and

determining the risk that the health of a neonate with respiratory distress syndrome will be jeopardized by weaning from a ventilator,

wherein the sample of a bodily fluid is further defined as a sample of a fluid selected from group consisting of lung secretions, saliva, nasal secretions, urine, and stool.

64. The method of paragraph 63, wherein the level of LPCAT1 in the sample of a bodily fluid is further defined as a copy number of cell free LPCAT1 mRNA per unit volume of a bodily fluid, as determined according to the method of paragraph 1.
65. The method of paragraph 63, wherein level of LPCAT1 in the sample of a bodily fluid is further defined as a level of LPCAT1 protein in the sample of a bodily fluid, as determined according to the method of paragraph 40.
66. The method of paragraph 63 further including the step of making a treatment recommendation, based the recommendation selected from the group including continuing the neonate on a ventilator and weaning the neonate from a ventilator.
67. Reagents for polymerase chain reaction (PCR) amplification of a target sequence in human LPCAT1, and the real-time PCR (RT-PCR) quantitation of the amplicon thereby produced, including a forward primer including an oligonucleotide with at least 95% identity with SEQ ID NO: 1 or the exact complement thereof, a reverse primer including an oligonucleotide with at least 95% identity with SEQ ID NO: 2 or the exact complement thereof, and a fluorescent reporter probe including an oligonucleotide including SEQ ID NO: 3 or the exact complement thereof
68. A biomarker for the assessments of fetal or neonatal lung maturation or of lung function of any individual, said marker comprising cell free mRNA encoding lysophospholipid acyltransferase 1 (LPCAT1), wherein the amount of LPCAT1 mRNA occurring in a sample is proportional to a level of fetal lung maturity.
69. The biomarker of paragraph 68, wherein said sample is selected from the group including a maternal serum sample, an amniotic fluid sample, a lung secretion sample, a saliva sample, a nasal secretion sample, a urine sample, and a stool sample.
70. A method for antenatally assessing fetal lung maturity, including the steps of:

collecting a sample of maternal serum;

measuring a level of lysophospholipid acyltransferase 1 (LPCAT1) protein in the sample of maternal serum;

comparing the level of LPCAT1 protein in the sample of maternal serum to a reference standard relating a level of LCAT1 protein to a level of fetal lung maturity;

determining the level of lung maturity of a fetus corresponding to the LPCAT1 protein in the sample of maternal serum according to the reference standard; and

assessing the level of lung maturity of a fetus.

71. The method of paragraph 70, wherein the measuring step is further defined as measuring a level of LPCAT1 protein by means of an immunological assay with an anti-LPCAT1 antibody.
72. The method of paragraph 71, wherein the immunological assay is selected from the group including an ELISA, a western blot, and a fluorescent bead based immunoassay.
73, The method of paragraph 71 wherein the level LPCAT1 protein in the sample of maternal is further defined as a weight LPCAT1 protein per cc of maternal serum.
74. The method of paragraph 70, wherein the measuring step is further defined as measuring a level of LPCAT1 protein by means of an assay of lysophospholipid acyltransferase activity.
75. The method of paragraph 74, wherein the level of LPCAT1 protein is further defined as a measure selected from the group consisting of a weight LPCAT1 protein per cc of maternal serum and a number of units of lysophospholipid acyltransferase activity per cc of maternal serum.
76. A method for assessing the risk of a fetus for developing neonatal respiratory distress syndrome, including the steps of:

collecting a sample of maternal serum;

measuring a level of lysophospholipid acyltransferase 1 (LPCAT1) protein in the sample of maternal serum;

comparing the level of LPCAT1 protein in the sample of maternal serum to a threshold level of LPCAT1 protein, wherein a level of LPCAT1 protein below the threshold level indicates risk that a fetus will develop neonatal respiratory distress syndrome; and

determining whether risk a fetus is at risk of neonatal respiratory distress syndrome.

77. The method of paragraph 76, wherein the measuring step is further defined as measuring a level of LPCAT1 protein by means of an immunological assay with an anti-LPCAT1 antibody.
78. The method of paragraph 77, wherein the immunological assay is selected from the group including an ELISA, a western blot, and a fluorescent bead based immunoassay.
79. The method of paragraph 77 wherein the level LPCAT1 protein in the sample of maternal serum is further defined as a weight LPCAT1 protein per cc of maternal serum.
80. The method of paragraph 76, wherein the measuring step is further defined as measuring a level of LPCAT1 protein by means of an assay lysophospholipid acyltransferase activity.
81. The method of paragraph 80, wherein the level of LPCAT1 protein is further defined as a measure selected from the group consisting of a weight LPCAT1 protein per cc of maternal serum and a number of units of lysophospholipid acyltransferase activity per cc of maternal serum.
82. The method of paragraph 76, wherein the threshold level of LPCAT1 protein is further defined as a level of LPCAT1 protein known to be correlated to a level of an amniotic fluid biomarker associated with fetal risk for developing neonatal respiratory distress syndrome.
83. The method of paragraph 82, wherein the amniotic fluid biomarker is further defined as lamellar body count (LBC), and the threshold level of maternal serum LPCAT1 protein is further defined as a level of maternal serum LPCAT1 protein known to be correlated with an amniotic LBC of 40,000-50,000 lamellar bodies/μl amniotic fluid.
84. The method of paragraph 83, wherein the threshold level of maternal serum LPCAT1 protein is further defined as a level of maternal serum LPCAT1 protein known to be correlated with an amniotic LBC of 50,000 lamellar bodies/μl maternal serum.
85. The method of paragraph 76, additionally including the steps of:

determining that the level of LPCAT1 protein in the sample of maternal serum is below the threshold level of known to indicate sufficient lung maturity; and

treating the fetus to reduce the likelihood of respiratory distress at parturition.

86. The method of paragraph 85, wherein the step of treating the fetus is further defined as administering corticosteroid treatments to its mother to accelerate fetal lung maturation.
87. The method of paragraph 85, wherein the step of treating the fetus is further defined as recommending against preterm delivery.
88. The method of paragraph 85 wherein the step of treating the fetus is further defined as administering gene therapy to the fetus.
89. The method of paragraph 88, wherein the step of administering gene therapy further includes the steps of administering to a fetus a sufficient quantity of an expression vector encoding LPCAT1 and inducing in the lung tissue of a fetus a sufficient expression of LPCAT1 to prevent respiratory distress upon parturition.
90. A method for determining the effect of a treatment to accelerate lung maturation in a fetus, including the steps of:

collecting a sample of maternal serum at each of a plurality successive time points, the first time point being either prior to or after the initiation of the treatment;

measuring a level of lysophospholipid acyltransferase 1 (LPCAT1) protein in the sample of maternal serum per unit volume of maternal serum at each of the successive time points over the course of the treatment, the level of LPCAT1 protein per unit volume of maternal serum indicating a level of lung maturation;

comparing the level of LPCAT1 protein per unit volume of maternal serum at each time point to the level of LPCAT1 protein per unit volume of maternal serum at its immediately preceding time point over the course of the treatment;

calculating the differences among levels of LPCAT1 protein per unit volume of maternal serum over the successive time points over the course of the treatment; and

determining the effect of a treatment to accelerate lung maturation in a fetus, with increasing levels of LPCAT1 protein per unit volume of maternal serum over the course of the treatment indicating the acceleration of fetal lung maturation.

91. The method of paragraph 90, further including the step of making a treatment recommendation based on the step of monitoring the effect of monitoring the effect of the treatment to accelerate lung maturation in a fetus, the recommendation selected from the group including continuation of the treatment, discontinuation of the treatment, and initiation of a different treatment.
92. A method for monitoring the progress of fetal lung development, including the steps of:

collecting a sample of maternal serum at each of a plurality successive time points during gestation;

measuring a level of lysophospholipid acyltransferase 1 (LPCAT1) protein per unit of maternal serum at each of the successive time points over the course of gestation, the level of LPCAT1 protein per unit volume of maternal serum indicating a level of lung maturation;

comparing the level of LPCAT1 protein per unit volume of maternal serum at each time point to the level of LPCAT1 protein per unit volume of maternal serum at its immediately preceding time point over the course of gestation;

calculating the differences among levels of LPCAT1 protein per unit over the successive time points over the course of gestation, wherein the differences represent the progress of fetal lung development; and

monitoring the progress of fetal lung development.

93. A method for assessing lung function in an individual, including the steps of:

collecting a sample of a bodily fluid from an individual, wherein the sample of a bodily fluid is further defined as a sample of a fluid selected from the group consisting of lung secretions, saliva, nasal secretions, urine, and stool

determining a level of lysophospholipid acyltransferase 1 (LPCAT1) in the sample of a bodily fluid;

comparing the level of LPCAT1 in the sample of a bodily fluid to a reference standard relating a level of LPCAT1 to a level of lung function;

determining the level of lung function of the individual corresponding to the level of LPCAT1 in the sample of a bodily fluid; and

assessing the level of lung function in the individual.

94. The method of paragraph 93, wherein the level of LPCAT1 in the sample of a bodily fluid is further defined as a copy number of cell free LPCAT1 mRNA per unit volume of a bodily fluid, as determined according to the method of paragraph 1.
95. The method of paragraph 93, wherein level of LPCAT1 in the sample of a bodily fluid is further defined as a level of LPCAT1 protein in the sample of a bodily fluid, as determined according to the method of paragraph 40.
96. A method for assessing the risk that the health of an individual in respiratory distress will be jeopardized by weaning from a ventilator, including the steps of:

collecting a sample of a bodily fluid from an individual, wherein the sample of a bodily fluid is further defined as a sample of a fluid selected from the group consisting of lung secretions, saliva, nasal secretions, urine, and stool;

determining a level of lysophospholipid acyltransferase 1 (LPCAT1) in the sample of a bodily fluid;

Comparing the level of LPCAT1 in the sample of a bodily fluid to a threshold level of LPCAT1, wherein a level of LPCAT1 below the threshold level indicates risk that the lung function of an individual is insufficient to support independent respiration; and

determining the risk that the health of an individual in respiratory distress will be jeopardized by weaning from a ventilator.

97. The method of paragraph 96, wherein the level of LPCAT1 in the sample of a bodily fluid is further defined as a copy number of cell free LPCAT1 mRNA per unit volume of a bodily fluid, as determined according to the method of paragraph 1.
98. The method of paragraph 96, wherein level of LPCAT1 in the sample of a bodily fluid is further defined as a level of LPCAT1 protein in the sample of a bodily fluid, as determined according to the method of paragraph 40.
99. The method of paragraph 96 further including the step of making a treatment recommendation, based the recommendation selected from the group including continuing the individual on a ventilator and weaning the individual from a ventilator.
100. A method for treating a patient bearing a fetus at risk of neonatal respiratory distress syndrome including the steps of

determining that a level of LPCAT1 expression in a sample of a body fluid is below a threshold level known to indicate sufficient lung maturity; and

administering a treatment selected from the group of treatments including corticosteroid treatments to accelerate lung maturation, gene therapy to increase the expression of LPCAT1 protein in fetal lung tissues, and recommending against preterm delivery.

101. The method of paragraph 100, wherein the body fluid is maternal serum or amniotic fluid.
102. The method of paragraph 101 wherein the level of LPCAT1 expression is further defined as a copy number of cell-free LPCAT1 mRNA per cc of the body fluid.
103. The method of paragraph 102, wherein the threshold level of LPCAT1 expression is defined as 100 copies of cell-free LPCAT1 mRNA per cc of the body fluid.
104. The method of paragraph 101, wherein the threshold level of LPCAT1 expression is further defined as a level selected from the group including a weight of LPCAT1 protein per cc of the body fluid and a number of units of lysophospholipid acyltransferase activity per cc of the body fluid.
105. The method of paragraph 104, wherein the threshold level of LPCAT1 expression is further defined as a level of LPCAT1 expression known to be correlated to a lamellar body count of 40,000-50,000 lamellar bodies/ul amniotic fluid.
106 A method for ventilation therapy including the steps of:

determining whether a level of LPCAT1 expression in a sample of a body fluid of an individual on a ventilator is below a threshold level known to indicate a risk that the health of the individual with respiratory distress syndrome will be jeopardized by weaning from a ventilator; and

recommending against the weaning of those individuals exhibiting a level of LPCAT1 expression that is below than the threshold level.

107. The method of paragraph 106, wherein the body fluid is selected from the group consisting of lung secretions, saliva, nasal secretions, urine, and stool.
108. The method of paragraph 107 wherein the level of LPCAT1 expression is further defined as a copy number of cell-free LPCAT1 mRNA per cc of the body fluid.
109. The method of paragraph 106, wherein the threshold level of LPCAT1 expression is further defined as a level selected from the group including a weight of LPCAT1 protein per cc of the body fluid and a number of units of lysophospholipid acyltransferase activity per cc of the body fluid.
110. An assay kit for determining a concentration of cell free lysophospholipid acyltransferase 1 (LPCAT1) mRNA in a sample of body fluid for the antenatal assessment of fetal lung maturity, including:

at least one nucleic acid reagent specifically hybridizable to at least one nucleic acid sequence encoding LPCAT1; and

a quantitation system for quantitating the hybridization of said nucleic acid reagent with said least one nucleic acid sequence encoding LPCAT1 and deriving a concentration value of LPCAT1 mRNA per unit volume of said sample of body fluid.

111. The assay kit of paragraph 110, wherein the body fluid is maternal serum or amniotic fluid.
112. The assay kit of paragraph 110 wherein said quantitation system includes a polymerase chain reaction (PCR), said at least one nucleic acid sequence encoding LPCAT1 is further defined as a cDNA transcribed from said cell free LPCAT1 mRNA, said at least one nucleic acid reagent is further defined as at least one oligonucleotide primer pair for amplifying said LPCAT1 cDNA, and said concentration value of cell free LPCAT1 mRNA is defined as a copy number value of cell free LPCAT1 mRNA per unit volume of the sample of body fluid.
113. The assay kit of paragraph 112 further including instructions for relating said copy number value of cell free LPCAT1 mRNA per unit volume to a level of fetal lung maturity.
114. The assay kit of paragraph 112, wherein said at least one oligonucleotide primer pair includes SEQ ID NO:1 and SEQ ID NO:2.
115. The assay kit of paragraph 113, wherein said PCR produces an amplified product including SEQ ID NO.4.
116. The assay kit of paragraph 112, further including reagents for reverse transcribing into cDNA the cell-free LPCAT1 mRNA in the sample of body fluid.
117. The assay kit of paragraph 112 wherein said PCR is real-time (RT)-PCR.
118. An assay kit for determining a concentration of cell free lysophospholipid acyltransferase 1 (LPCAT1) mRNA in a sample of body fluid for the assessment of a risk of a fetus for developing neonatal respiratory distress syndrome, including:

at least one nucleic acid reagent specifically hybridizable to at least one nucleic acid sequence encoding LPCAT1; and

a quantitation system for quantitating the hybridization of said nucleic acid reagent with said least one nucleic acid sequence encoding LPCAT1 and deriving a concentration value of LPCAT1 mRNA per unit volume of said sample of body fluid.

119. The assay kit of paragraph 118, wherein the body fluid is maternal serum or amniotic fluid.
120. The assay kit of paragraph 118 wherein said quantitation system includes a polymerase chain reaction (PCR), said at least one nucleic acid sequence encoding LPCAT1 is further defined as a cDNA transcribed from said cell free LPCAT1 mRNA, said at least one nucleic acid reagent is further defined as at least one oligonucleotide primer pair for amplifying said LPCAT1 cDNA, and said concentration value of cell free LPCAT1 mRNA is defined as a copy number value of cell free LPCAT1 mRNA per unit volume of the sample of body fluid.
121. The assay kit of paragraph 3 further including instructions for relating said copy number value of cell free LPCAT1 mRNA per unit volume to a risk of a fetus for developing neonatal respiratory distress syndrome.
122. The assay kit of paragraph 120, wherein said at least one oligonucleotide primer pair includes SEQ ID NO:1 and SEQ ID NO:2.
123. The assay kit of paragraph 121, wherein said PCR produces an amplified product including SEQ ID NO.4.
124. The assay kit of paragraph 120, further including reagents for reverse transcribing into cDNA the cell-free LPCAT1 mRNA in the sample of body fluid.
125. The assay kit of paragraph 124 wherein said PCR is real-time (RT)-PCR.
126. An assay kit for determining a concentration of cell free lysophospholipid acyltransferase 1 (LPCAT1) mRNA in a plurality of serial samples of a body fluid to determine the effect of a treatment to accelerate lung maturation in a fetus, including:

at least one nucleic acid reagent specifically hybridizable to at least one nucleic acid sequence encoding LPCAT1; and

a quantitation system for quantitating the hybridization of said nucleic acid reagent with said least one nucleic acid sequence encoding LPCAT1 and deriving a concentration value of LPCAT1 mRNA per unit volume of a first sample of the body fluid; said first sample being obtained at a first time point in relation to the treatment and of a second sample of the body fluid, said second sample being obtained at a second time point in relation to the treatment.

127. The assay kit of paragraph 126, wherein the body fluid is maternal serum or amniotic fluid.
128. The assay kit of paragraph 126, wherein said first time point is a time point prior to the initiation of the treatment, and said second time point is a time point subsequent to the initiation of the treatment.
129. The assay kit of paragraph 126 further including instructions for relating a difference between said concentration values of LPCAT1 of said first sample and of said second sample to an effect of the treatment on fetal lung maturity.
130. The assay kit of paragraph 126 wherein said quantitation system includes a polymerase chain reaction (PCR), said at least one nucleic acid sequence encoding LPCAT1 is further defined as a cDNA transcribed from said cell free LPCAT1 mRNA, said at least one nucleic acid reagent is further defined as at least one oligonucleotide primer pair for amplifying said LPCAT1 cDNA, and said concentration value of cell free LPCAT1 mRNA is defined as a copy number value of cell free LPCAT1 mRNA per unit volume of the sample of body fluid.
131. The assay kit of paragraph 130, wherein said at least one oligonucleotide primer pair includes SEQ ID NO:1 and SEQ ID NO:2.
132. The assay kit of paragraph 131, wherein said. PCR produces an amplified product including SEQ ID NO 4.
133. The assay kit of paragraph 130 further including reagents for reverse transcribing into cDNA the cell-free LPCAT1 mRNA in the sample of the body fluid.
134. An assay kit for determining a concentration of cell free lysophospholipid acyltransferase 1 (LPCAT1) mRNA in a plurality of serial samples of a body fluid to monitor the progress of lung maturation in a fetus, including:

at least one nucleic acid reagent specifically hybridizable to at least one nucleic acid sequence encoding LPCAT1;

and a quantitation system for quantitating the hybridization of said nucleic acid reagent with said least one nucleic acid sequence encoding LPCAT1 and deriving a concentration value of LPCAT1 mRNA per unit volume of a first sample of the body fluid, said first sample being obtained at a first time point during gestation and of a second sample of the body fluid, said second sample being obtained at a second time point during gestation.

135. The assay kit of paragraph 134, wherein the body fluid is maternal serum or amniotic fluid.
136. The assay kit of paragraph 134 further including instructions for relating a difference between said concentration values of LPCAT1 of said first sample and of said second sample to the progress of fetal lung maturation.
137. The assay kit of paragraph 134 wherein said quantitation system includes a polymerase chain reaction (PCR), said at least one nucleic acid sequence encoding LPCAT1 is further defined as a cDNA transcribed from said cell free LPCAT1 mRNA, said at least one nucleic acid reagent is further defined as at least one oligonucleotide primer pair for amplifying said LPCAT1 cDNA, and said concentration value of cell free LPCAT1 mRNA is defined as a copy number value of cell free LPCAT1 mRNA per unit volume of the sample of body fluid.
138. The assay kit of paragraph 137, wherein said at least one oligonucleotide primer pair includes SEQ ID NO:1 and SEQ ID NO:2.
139. The assay kit of paragraph 138, wherein said PCR produces an amplified product including SEQ ID NO 4.
140. The assay kit of paragraph 138, further including reagents for reverse transcribing into cDNA the cell-free LPCAT mRNA in the sample of the body fluid.
141. The assay kit of paragraph 138 wherein said PCR is real-time (RT)-PCR.
142. An assay kit for determining a concentration of cell free lysophospholipid acyltransferase 1 (LPCAT1) mRNA in a sample of body fluid for the assessment of lung maturity in a neonate, including:

at least one nucleic acid reagent specifically hybridizable to at least one nucleic acid sequence encoding LPCAT1;

and a quantitation system for quantitating the hybridization of said nucleic acid reagent with said least one nucleic acid sequence encoding LPCAT1 and deriving a concentration value of LPCAT1 mRNA per unit volume of said sample of the body fluid.

143 The assay kit of paragraph 142, wherein the body fluid is selected from the group consisting of lung secretions, saliva, nasal secretions, urine, and stool.
144. The assay kit of paragraph 142 wherein said quantitation system includes a polymerase chain reaction (PCR), said at least one nucleic acid sequence encoding LPCAT1 is further defined as a cDNA transcribed from said cell free LPCAT1 mRNA, said at least one nucleic acid reagent is further defined as at least one oligonucleotide primer pair for amplifying said LPCAT1 cDNA, and said concentration value of cell free LPCAT1 mRNA is defined as a copy number value of cell free LPCAT1 mRNA per unit volume of the sample of the body fluid.
145. The assay kit of paragraph 144 further including instructions for relating said copy number value of cell free LPCAT1 mRNA per unit volume to a level of lung maturity.
146. The assay kit of paragraph 144, wherein said at least one oligonucleotide primer pair includes SEQ ID NO:1 and SEQ ID NO:2.
147. The assay kit of paragraph 145, wherein said PCR produces an amplified product including SEQ ID NO 4.
148. The assay kit of paragraph 142, further including reagents for reverse transcribing into cDNA the cell-free LPCAT1 mRNA in the sample of the body fluid.
149. The assay kit of paragraph 146 wherein said PCR is real-time (RT)-PCR.
150. An assay kit for determining a concentration of lysophospholipid acyltransferase 1 (LPCAT1) mRNA in a sample of body fluid of an individual, for the assessment of a risk that the health of the individual will be jeopardized by weaning from a ventilator, including:

at least one nucleic acid reagent specifically hybridizable to at least one nucleic acid sequence encoding LPCAT1;

and quantitation means for quantitating the hybridization of said nucleic acid reagent with said least one nucleic acid sequence encoding LPCAT1 and deriving a concentration value of LPCAT1 mRNA per unit volume of said sample of the body fluid.
151. The assay kit of paragraph 150, wherein the body fluid is selected from the group consisting of lung secretions, saliva, nasal secretions, urine, and stool.
152. The assay kit of paragraph 150 wherein said quantitation means includes a polymerase chain reaction (PCR), said at least one nucleic acid sequence encoding LPCAT1 is further defined as a cDNA transcribed from said cell free LPCAT1 mRNA, said at least one nucleic acid reagent is further defined as at least one oligonucleotide primer pair for amplifying said LPCAT1 cDNA, and said concentration value of cell free LPCAT1 mRNA is defined as a copy number value of cell free LPCAT1 RNA per unit volume of the sample of the body fluid.
153. The assay kit of paragraph 1 further including instructions for relating said copy number value of cell free LPCAT1 mRNA per unit volume to a level of risk that the health of an individual will be jeopardized by weaning from a ventilator.
154. The assay kit of paragraph 152, wherein said at least one oligonucleotide primer pair includes SEQ ID NO:1 and SEQ ID NO:2.
155. The assay kit of paragraph 154, wherein said PCR produces an amplified product including SEQ ID NO 4.
156. The assay kit of paragraph 152, further including reagents for reverse transcribing into cDNA the cell-free LPCAT1 mRNA in the sample of the body fluid.
157. The assay kit of paragraph 154 wherein said PCR is real-time (RT)-PCR.
158. An assay kit for quantitating cell free lysophospholipid acyltransferase 1 (LPCAT1) mRNA in a sample of body fluid of an individual for the assessment of lung function in the individual, including:

at least one nucleic acid reagent specifically hybridizable to at least one nucleic acid sequence encoding LPCAT1; and

quantitation means for quantitating the hybridization of said nucleic acid reagent with said least one nucleic acid sequence encoding LPCAT1 and deriving a concentration value of LPCAT1 mRNA per unit volume of said sample of the body fluid.

159. The assay kit of paragraph 158, wherein the body fluid is selected from the group consisting of lung secretions, saliva, nasal secretions, urine, and stool.
160. The assay kit of paragraph 158 wherein said quantitation means includes a polymerase chain reaction (PCR), said at least one nucleic acid sequence encoding LPCAT1 is further defined as a cDNA transcribed from said cell free LPCAT1 mRNA, said at least one nucleic acid reagent is further defined as at least one oligonucleotide primer pair for amplifying said LPCAT1 cDNA, and said concentration value of cell free LPCAT1 mRNA is defined as a copy number value of cell free LPCAT1 mRNA per unit volume of the sample of the body fluid.
161. The assay kit of paragraph 160 further including instructions for relating said copy number value of cell free LPCAT1 mRNA per unit volume to a level of lung function.
162. The assay kit of paragraph 160, wherein said at least one oligonucleotide primer pair includes SEQ ID NO:1 and SEQ ID NO:2.
163. The assay kit of paragraph 162, wherein said PCR produces an amplified product including SEQ ID NO 4.
164. The assay kit of paragraph 160, further including reagents for reverse transcribing into cDNA the cell-free LPCAT1 mRNA in the sample of the body fluid.
165. The assay kit of paragraph 162 wherein said. PCR is real-time (RT)-PCR.
166. An immunoassay kit for quantitating lysophospholipid acyltransferase 1 (LPCAT1) protein in a sample of a body fluid, for the antenatal assessment of fetal lung maturity, including: an immunosensor for quantitating an immunoreaction between said at least one antibody and LPCAT1 protein, said immunosensor providing a concentration value of LPCAT1 protein per unit volume of the sample of body fluid; and instructions for relating said concentration value of LPCAT1 protein per unit volume of the sample of body fluid to a level of fetal lung maturity.
167. The immunoassay kit of paragraph 166, wherein the body fluid is maternal serum of amniotic fluid.
168. The immunoassay kit of paragraph 166, wherein said immunosensor is further defined as a sandwich immunoassay.
169. The immunoassay kit of paragraph 168, wherein said at least one antibody specifically reactive with LPCAT1 protein includes a capture antibody specifically reactive with a first epitope of LPCAT1 and a detection antibody specifically reactive with a second epitope of LPCAT1.
170. An immunoassay kit for quantitating lysophospholipid acyltransferase 1 (LPCAT1) protein in a sample of a body fluid for the antenatal assessment of risk of a fetus for developing neonatal respiratory distress syndrome, including:

an immunosensor for quantitating an immunoreaction between said at least one antibody and LPCAT1 protein, said immunosensor providing a concentration value of LPCAT1 protein per unit volume of the sample of body fluid, and

instructions for relating said concentration value of LPCAT1 protein per unit volume of said sample of body fluid to a risk of neonatal respiratory distress syndrome.

171. The immunoassay kit of paragraph 170, wherein the body fluid is maternal serum of amniotic fluid.
172. The immunoassay kit of paragraph 170, wherein said immunosensor is further defined as a sandwich immunoassay.
173. The immunoassay kit of paragraph 172, wherein said at least one antibody specifically reactive with LPCAT1 protein includes a capture antibody specifically reactive with a first epitope of LPCAT1 and a detection antibody specifically reactive with a second epitope of LPCAT1.
174. An immunoassay kit for quantitating lysophospholipid acyltransferase 1 (LPCAT1) protein in a plurality of serial samples of a body fluid to determine the effect of a treatment to accelerate lung maturation in a fetus, including:

an immunosensor for quantitating an immunoreaction between said at least one antibody and LPCAT1 protein, said immunosensor providing a concentration value of LPCAT1 protein per unit volume of a first sample of the body fluid, said first sample being obtained at a first time point in relation to the treatment and of a second sample of the body fluid, said second sample being obtained at a second time point in relation to the treatment, and

instructions for relating a difference between said concentration values of LPCAT1 of said first sample and of said second sample to an effect of the treatment on fetal lung maturity.

175. The immunoassay kit of paragraph 174, wherein the body fluid is maternal serum or amniotic fluid.
176. The immunoassay kit of paragraph 174, wherein said first time point is a time point prior to the initiation of the treatment, and said second time point is a time point subsequent to the initiation of the treatment.
177. The immunoassay kit of paragraph 174, wherein said immunosensor is further defined as a sandwich immunoassay.
178. The immunoassay kit of paragraph 177, wherein said at least one antibody specifically reactive with LPCAT1 protein includes a capture antibody specifically reactive with a first epitope of LPCAT1 and a detection antibody specifically reactive with a second epitope of LPCAT1.
179. An immunoassay kit for quantitating lysophospholipid acyltransferase 1 (LPCAT1) protein in a plurality of serial samples of a body fluid to monitor the progress of lung maturation in a fetus, including:

an immunosensor for quantitating an immunoreaction between said at least one antibody and LPCAT1 protein, said immunosensor providing a concentration value of LPCAT1 protein per unit volume of a first sample of the body fluid, said first sample being obtained at a first time point during gestation, and of a second sample of the body fluid, said second sample being obtained at a second time during gestation, and

instructions for relating a difference between said concentration values of LPCAT1 of said first sample and of said second sample to the progress of fetal lung maturation.

180. The immunoassay kit of paragraph 179, wherein the body fluid is maternal serum or amniotic fluid.
181. The immunoassay kit of paragraph 179, wherein said immunosensor is further defined as a sandwich immunoassay.
182. The immunoassay kit of paragraph 181, wherein said at least one antibody specifically reactive with LPCAT1 protein includes a capture antibody specifically reactive with a first epitope of LPCAT1 and a detection antibody specifically reactive with a second epitope of LPCAT1,
183. An immunoassay kit for quantitating lysophospholipid acyltransferase 1 (LPCAT1) protein in a sample of a body fluid for the assessment of lung maturity in a neonate, including:

an immunosensor for quantitating an immunoreaction between said at least one antibody and LPCAT1 protein, said immunosensor providing a concentration value of LPCAT1 protein per unit volume of the sample of body fluid, and

instructions for relating said concentration value of LPCAT1 protein per unit volume of the sample of body fluid to a level of lung maturity.

184. The immunoassay kit of paragraph 183, wherein the body fluid is selected from the group consisting of lung secretions, saliva, nasal secretions, urine, and stool.
185. The immunoassay kit of paragraph 183, wherein said immunosensor is further defined as a sandwich immunoassay.
186. The immunoassay kit of paragraph 185, wherein said at least one antibody specifically reactive with LPCAT1 protein includes a capture antibody specifically reactive with a first epitope of LPCAT1 and a detection antibody specifically reactive with a second epitope of LPCAT1.
187. An immunoassay kit for quantitating lysophospholipid acyltransferase 1 (LPCAT1) protein in a sample of a body fluid of an individual for the assessment of risk that the health of the individual will be jeopardized by weaning from a ventilator, including:

an immunosensor for quantitating an immunoreaction between said at least one antibody and LPCAT1 protein, said immunosensor providing a concentration value of LPCAT1 protein per unit volume of the sample of the body fluid; and

instructions for relating said concentration value of LPCAT1 protein per unit volume of the sample of body fluid to a risk that the health of an individual will be jeopardized by weaning from a ventilator.

188. The immunoassay kit of paragraph 187, wherein the body fluid is selected from the group consisting of lung secretions, saliva, nasal secretions, urine, and stool.
189. The immunoassay kit of paragraph 187, wherein said immunosensor is further defined as a sandwich immunoassay.
190. The immunoassay kit of paragraph 189, wherein said at least one antibody specifically reactive with LPCAT1 protein includes a capture antibody specifically reactive with a first epitope of LPCAT1 and a detection antibody specifically reactive with a second epitope of LPCAT1.
191. An immunoassay kit for quantitating lysophospholipid acyltransferase 1 (LPCAT1) protein in a sample of body fluid of an individual for the assessment of lung function in the individual, including:

an immunosensor for quantitating an immunoreaction between said at least one antibody and LPCAT1 protein, said immunosensor providing a concentration value of LPCAT1 protein per unit volume of the sample of the body fluid; and

instructions for relating said concentration value of LPCAT1 protein per unit volume of the sample of body fluid to a level of lung function.

192. The immunoassay kit of paragraph 191, wherein the body fluid is selected from the group consisting of lung secretions, saliva, nasal secretions, urine, and stool.
193. The immunoassay kit of paragraph 191, wherein said immunosensor is further defined as a sandwich immunoassay.
194. The immunoassay kit of paragraph 193, wherein said at least one antibody specifically reactive with LPCAT1 protein includes a capture antibody specifically reactive with a first epitope or LPCAT1 and a detection antibody specifically reactive with a second epitope of LPCAT1.
195. A method of detecting fetal LPCAT1 mRNA, the method comprising contacting maternal serum with LPCAT1-specific primers and amplifying the LPCAT1 mRNA.

### Example 1

### The Biomarkers Maternal Serum Fetal LPCAT1 mRNA and Amniotic Fluid Fetal LPCAT1 mRNA Correlate Closely with Amniotic Fluid Lamellar Body Count (LBC) in the Assessment of Fetal Lung Maturation

Materials and Methods

Patient Populations

Amniotic fluid and blood samples were obtained from 29 patients in the third trimester undergoing amniocentesis for obstetrical indications. For control purposes, blood was also obtained from four non-pregnant individuals. Approximately 5 cc of venous blood and 25 cc of amniotic fluid were collected.

RT-PCR Assay

A robust Taqman type PCR assay was developed for the amplification of a target sequence of human LPCAT1 cDNA. The validity of the assay was confirmed by a close exponential relationship between threshold cycle number and plasmid copy number. No amplification was seen in the absence of plasmid DNA or in the presence of unrelated plasmid DNA. Agarose gel analysis of PCR samples detected a single band of appropriate size (not shown).

For the assay of LPCAT1 in patient tissues supernatant and cells from amniotic fluid and cell serum were obtained from pregnant individuals. mRNA was isolated from cells using RNeasy kits (Qiagen) per manufacturer's instructions. mRNA was isolated from amniotic fluid and serum using Trizol reagent (Invitrogen) followed by secondary isolation using RNeasy kits. Samples of mRNA were quantified and purity confirmed by spectrophotometry, and then cDNA was synthesized by reverse transcription. cDNA samples were subjected to RT-PCR to quantitate LPCAT1 expression. The target amplicon was a fragment of human LPCAT1 including SEQ ID NO: 4. The forward primer was an oligonucleotide of SEQ ID NO: 1 and the reverse primer was an oligonucleotide of SEQ ID NO: 2. Taqman chemistry was used, and the probe for detecting the amplicon was a Texas Red Black Hole Quencher labeled probe of SEQ ID NO: 3.

Lamellar Body Count (LBC) Assay

A minimum of 1.0 ml of amniotic fluid was collected via trans-abdominal amniocentesis or from a vaginal pool. The amniotic fluid samples were not centrifuged and were either held at ambient temperature for up eight hours or stored up to 48 hrs at 2-8 C.° before testing. Amniotic fluid samples were mixed by inversion a minimum of ten times and approximately 1.0 ml of fluid was poured into a clear test tube. Samples containing meconium or mucus, or a hematocrit >1% were not used in this study. The samples were then counted in a Sysmex XE2100 automated cell counter in manual mode and analyzed in platelet analysis mode. Only samples wherein the platelet impedence histogram showed no interference, and the curve returned to at least 20% of baseline were included in this study. Results were expressed in terms of 10³ lamellar bodies/μl.

Results

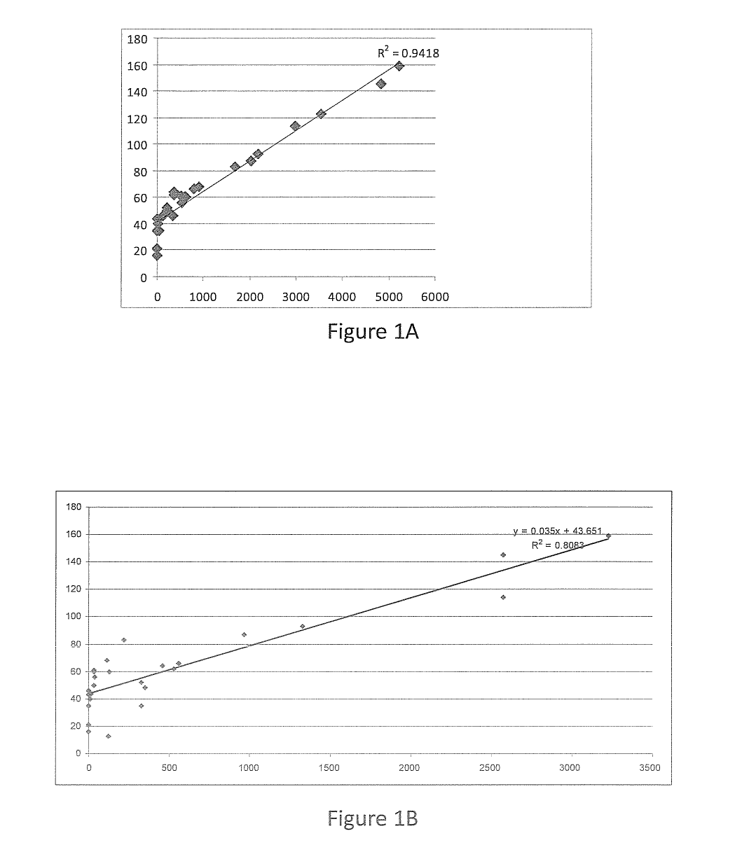

LPCAT1 expression was detected in both maternal blood and amniotic fluid. LPCAT1 expression increased with increasing lung maturity as assessed by LBC counts in amniotic fluid. That is, there was with a 12 fold absolute difference detected in amniotic fluid with the highest and lowest LBC counts. Importantly, there was a close correlation between LPCAT1 expression in amniotic fluid and LBC in amniotic fluid (R²=0.94, p<0.01) (FIG. 1A) and between LPCAT1 expression in maternal serum and LBC in amniotic fluid (R²=0.80, p<0.07) (FIG. 1B).

Adult individuals also express LPCAT1 in lung and various other tissues, so it was necessary to determine whether most or all of the measurable LPCAT1 measured in maternal serum was derived from fetal sources. Serum was collected from four non-pregnant individuals and processed as described above. LPCAT1 amplification was detected in every individual, but in every case it corresponded to less than 5 copies of plasmid DNA per cc of serum. In contrast, the serum of pregnant individuals contained between 4 and 3227 copies of LPCAT1 mRNA. The results show that the maternal serum LPCAT1 mRNA measured by RT-PCR was essentially attributable to fetal LPCAT1 mRNA.

Discussion.

The results represent the first finding that cell fetal LPCAT1 mRNA is present at detectable levels in maternal serum. They also represent the first finding of a close correlation between the expression of serum fetal LPCAT1 mRNA and fetal lung maturity, as indicated by the close correlation between LBC values and copy number values of LPCAT1 mRNA in maternal serum. The results also represent the first finding, that levels of the cell free LPCAT1 mRNA in amniotic fluid is also closely correlated with LBC values and this with fetal lung maturity. In summary, the results demonstrate that both maternal serum fetal LPCAT1 mRNA and amniotic fluid fetal LPCAT1 are useful markers of fetal lung maturation.

### REFERENCES

ACOG (American College of Obstetricians and Gynecologists) Committee on obstetrical practice. Antenatal corticosteroid therapy for fetal maturation. Committee Opinion No. 475. Obstet. Gynecol 117: 422-424 (2011).
Akolekar R, Farkas D H, VanAgtmael A L, Bombard A T, Nicolaides K H. Fetal sex determination using circulating cell-free fetal DNA (ccffDNA) at 11 to 13 weeks of gestation. Prenat. Diag. 30: 918-923 (2010).
Ausubel et al. (eds) Current protocols in molecular biology, John Wiley & Sons, New York, N.Y., 1994-2013.
Bridges J P, Ikegami M, Brilli L L, Chen X, Mason R J, Shannon J M. LPCAT1 regulates surfactant phospholipid synthesis and is required for transitioning to air breathing in mice. J Clin Invest. 120: 1736-1748 (2010).
Eisenthal R and Danson R J, eds. Enzyme assays: a practical approach. (Oxford University Press, New York, 1992).
Frutos-Vivar F, Esteban A. When to wean from a ventilator: An evidence-based strategy. Cleveland Clinic J. Med. 70: 389-400 (2003).
Fan H C, Blumenfeld Y J, Chitkara U, Hudgins L, Quake S R. Noninvasive diagnosis of fetal aneuploidy by shotgun sequencing DNA from maternal blood. PNAS 105: 16266-16271 (2008).
Grenache D G, Gronowski A M. Fetal lung maturity. Clin. Biochem. 39: 1-10 (2006)
Harayama T Shindou H, Shimizu T. Biosynthesis of phosphatidylcholine by human lysophosphatidylcholine acyltransferase. J. Lipid Res. 50: 1824-1831 (2009).
Mishell and Shiigi (eds), Selected Methods in Cellular Immunology, W.H. Freeman and Co., New York (1980).
Neerhof M G, Dohnal J C, Ashwood E R, Lee I-S, Anceschi M M. Lamellar body counts: A consensus on protocol. Obst. Gynecol: 97:318-320 (2001).
Nakanishi H. Shindou H, Hishikawa D, Harayama. T, Ogasawara R, Suwabe A Taguchi R, Shimizu T M. Cloning and Characterization of Mouse Lung-type Acyl-CoA:Lysophosphatidylcholine Acyltransferase 1 (LPCAT1): Expression in alveolar type II cells and possible involvement surfactant production J. Biol. Chem, 2006 281: 20140-20147.
Sambrook et al, Molecular Cloning: A Laboratory Manual, Cold Springs Harbor, New York, 1989.
Stites et al. (eds). Basic and Clinical Immunology (8th Edition), Appleton & Lange, Norwalk, Conn. (1994)
Wataganara T, LeShane E S, Chen A Y, Borgatta L, Peter I, Johnson K L, Bianchi D W. Plasma γ-Globin gene expression suggests that fetal hematopoietic cells contribute to the pool of circulating cell-free fetal nucleic acids during pregnancy. Clin Chem 50: 689-693 (2004).

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

SEQUENCE LISTING

| SEQ ID NO: 1 | AGGATTCTCGCAGGAAAACAG |
|---|---|
| SEQ ID NO: 2 | TTGAAGGTAATTAGGCAGGTCC |
| SEQ ID NO: 3 | AGAAATCAAGAGACGGGCGCAGT |
| SEQ ID NO: 4 | TCAAGATCAT CAGCAATGCC TCCTGCACCA CCAACTGCTT AGCACCCCTG GCCAAGGTCA |
| | TCCATGACAA CTTTGGTATC GTGGAAGGAC TCATGACCAC AGTCCAT |
| SEQ ID NO: 5 | AGCCTCAAGATCATCAGCAATG |
| SEQ ID NO: 6 | ATGGACTGTGGTCATGAGTCCTT |
| SEQ ID NO: 7 | CCAACTGCTTAGCACCCC |
| SEQ ID NO: 8: | ATGGGGAAGG TGAAGGTCGG AGTCAACGGA TTTGGTCGTA TTGGGCGCCT GGTCACCAGG |
| 61 | GCTGCTTTTA ACTCTGGTAA AGTGGATATT GTTGCCATCA ATGACCCCTT CATTGACCTC |
| 121 | AACTACATGG TTTACATGTT CCAATATGAT TCCACCCATG GCAAATTCCA TGGCACCGTC |
| 181 | AAGGCTGAGA ACGGGAAGCT TGTCATCAAT GGAAATCCCA TCACCATCTT CCAGGAGCGA |
| 241 | GATCCCTCCA AAATCAAGTG GGGCGATGCT GGCGCTGAGT ACGTCGTGGA GTCCACTGGC |
| 301 | GTCTTCACCA CCATGGAGAA GGCTGGGGCT CATTTGCAGG GGGGAGCCAA AAGGGTCATC |
| 361 | ATCTCTGCCC CCTCTGCTGA CGCCCCCATG TTCGTCATGG GTGTGAACCA TGAGAAGTAT |
| 421 | GACAACAGCC TCAAGATCAT CAGCAATGCC TCCTGCACCA CCAACTGCTT AGCACCCCTG |
| 481 | GCCAAGGTCA TCCATGACAA CTTTGGTATC GTGGAAGGAC TCATGACCAC AGTCCATGCC |
| 541 | ATCACTGCCA CCCAGAAGAC TGTGGATGGC CCCTCCGGGA AACTGTGGCG TGATGGCCGC |
| 601 | GGGGCTCTCC AGAACATCAT CCCTGCCTCT ACTGGCGCTG CCAAGGCTGT GGGCAAGGTC |
| 661 | ATCCCTGAGC TGAACGGGAA GCTCACTGGC ATGGCCTTCC GTGTCCCCAC TGCCAACGTG |
| 721 | TCAGTGGTGG ACCTGACCTG CCGTCTAGAA AAACCTGCCA AATATGATGA CATCAAGAAG |
| 781 | GTGGTGAAGC AGGCGTCGGA GGGCCCCCTC AAGGGCATCC TGGGCTACAC TGAGCACCAG |
| 841 | GTGGTCTCCT CTGACTTCAA CAGCGACACC CACTCCTCCA CCTTCGACGC TGGGGCTGGC |
| 901 | ATTGCCCTCA ACGACCACTT TGTCAAGCTC ATTTCCTGGT ATGACAACGA ATTTGGCTAC |
| 961 | AGCAACAGGG TGGTGGACCT CATGGCCCAC ATGGCCTCCA AGGAGTAA |

SEQ ID NO: 9 LPCAT1 mRNA sequence NCBI Ref Seq: NM_024830

| | |
|---|---|
| 1 | gcccgctcca gccgccgcgc atcctcggcc cgcgccccga gacccgcgcc cagctagccc |
| 61 | cggccccgct cggcgcccca ggcagctcgg ctgcgctcgc cgcgggacgg cgcggccatg |
| 121 | aggctgcggg gatgcggacc ccgggccgcc cctgcctcca gcgcaggggc cagcgacgct |
| 181 | cggctgctgg cgcccccggg gcggaacccc ttcgtgcacg agctgcgcct cagcgccctg |
| 241 | cagaaggccc aggtggccct catgacactg acgctcttcc cggtccggct cctggttgcc |
| 301 | gctgccatga tgctgctggc ctggcccctc gcacttgtcg catccctggg ctctgcggag |
| 361 | aaggaacccg agcagccccc ggccctgtgg aggaaggttg tggacttcct gctgaaggcc |
| 421 | atcatgcgca ccatgtggtt cgccggcggc ttccaccggg tggccgtgaa ggggcggcag |
| 481 | gcgctgccca ccgaggcggc catcctcacg ctcgcgcctc actcgtccta cttcgacgcc |
| 541 | atccctgtga ccatgacgat gtcctccatc gtgatgaagg cagagagcag agacatcccg |
| 601 | atctggggaa ctctgatcca gtatatacgg cctgtgttcg tgtcccggtc agaccaggat |
| 661 | tctcgcagga aaacagtaga agaaatcaag agacgggcgc agtccaacgg aaagtggcca |
| 721 | cagataatga tttttccaga aggaacttgt acaaacagga cctgcctaat taccttcaaa |
| 781 | cctggtgcat tcatccctgg agcgcccgtc cagcctgtgg ttttacgata tccaaataaa |
| 841 | ctggacacca tcacatggac gtggcaagga cctggagcgc tggaaatcct gtggctcacg |
| 901 | ctgtgtcagt ttcacaacca agtggaaatc gagttccttc ctgtgtacag cccttctgag |
| 961 | gaggagaaga ggaaccccgc gctgtatgcc agcaacgtgc ggcgagtcat ggccgaggcc |
| 1021 | ttgggtgtct ccgtgactga ctacacgttc gaggactgcc agctggccct ggcggaagga |
| 1081 | cagctccgtc tccccgctga cacttgcctt ttagaatttg ccaggctcgt gcggggcctc |
| 1141 | gggctaaaac cagaaaagct tgaaaaagat ctggacagat actcagaaag agccaggatg |
| 1201 | aagggaggag agaagatagg tattgcggag tttgccgcct ccctggaagt ccccgtttct |
| 1261 | gacttgctgg aagacatgtt ttcactgttc gacgagagcg gcagcggcga ggtggacctg |
| 1321 | cgagagtgtg tggttgccct gtctgtcgtc tgccggccgg cccggaccct ggacaccatc |
| 1381 | cagctggctt tcaagatgta cggagcgcaa gaggacggca gcgtcggcga aggtgacctg |
| 1441 | tcctgcatcc tcaagacggc cctgggggtg gcagagctca ccgtgaccga cctattccga |
| 1501 | gccattgacc aagaggagaa ggggaagatc acattcgctg acttccacag gtttgcagaa |
| 1561 | atgtaccctg ccttcgcaga ggaatacctg tacccggatc agacacattt cgaaagctgt |
| 1621 | gcagagacct cacctgcgcc aatcccaaac ggcttctgtg ccgatttcag cccggaaaac |
| 1681 | tcagacgctg ggcggaagcc tgttcgcaag aagctggatt aggacccagg gttgcggaga |
| 1741 | gacgcggccc ctcccgcgtg gacatcaccg ccatgagcct ctttgcgagt gacctctggg |
| 1801 | ctccgctcct cactcctgct gtacaggcac tgtcttcagc ccgagttcca ggggcctcgg |
| 1861 | gggctgtttg tatcttgttc ctttgtgaag tgtgttgcag aaccgacgct tactgtgcga |
| 1921 | gaatcggagg gcgcgcacgc ggatcccccg cctggcctgg accccgtggg gtcaggttcc |
| 1981 | ctgccgggcg gggggcaccg gtgccgcccc gtgttctccc acggggccct ggtttcgagt |
| 2041 | ctctgtcaca gcctcttccg gcggcagcgt gcaccgggcg ggcctccgtg cacactcagc |
| 2101 | acacgcctgc cacacagcgt gcgcttgcgt gtcactctgg cacgaaacct gtctgcctct |
| 2161 | gtggatccac agcctggcag agccgagccg tcacctgatt tttcagtgtt tctacctgtg |
| 2221 | tgctggagct catgagtatt ttataaactc catttaggta cttcaggaaa catgcagcat |
| 2281 | tttttaaaaa atgaaaattg tttttctact tcatttttcc ttttagagtc aaaggatatt |
| 2341 | tatttatagg cctttttttt tttaatatag aatctgaggc tgtttgggct ttgacttaaa |
| 2401 | tttccatcag gcctctctcc agcaggtaat ccctctcctt ccgctgggtc ccctggggag |
| 2461 | gtgtgaactc aagggcctag ccccaaaaca ctttttctgc ttttcttaat ccttttccag |
| 2521 | tcccctcttt ttttataaac gttggcagtt tgatgtttct gtttcggcat aacgtaatcc |
| 2581 | atttcactgt agcctaaact ccagtccgag gttggatatt gttcaaatga gcagggcccg |
| 2641 | agctggaagc gcaaggcagc cgccgccgtg ccgctcctcc cttgccctca ggccaggtcc |
| 2701 | ctgctggaag cggctgcatc ttcctgtcag ccctggtttc catggtgact ggcgtcacgc |
| 2761 | agccacccga gtatggctga ccttcctgca gagagaggag ccgcagtctt ttgcttgtgg |
| 2821 | aaggagacgc tgggctgtgc ggtgcggagg gtgatgagga tgtctggtga cagccgtgcg |
| 2881 | gacaccactc ctctctgcag cactgcctcc cagcgccagg gtcgcgggca catcccactg |
| 2941 | agagcggggg tcctgcccca tcttagagtc aaaggcagag gggcttccag gccctggatg |
| 3001 | gggtattttg gtgtcacctg aagtccctct gacatcacct tgtttcatca ttttttatga |
| 3061 | cagaattaga aacccatcct tcaagcacaa taatcatcac agacttgagt ttgcttccta |
| 3121 | aagcaaaggc tccgggtttg tttggaaaat ttttttgatt tctgaaatga attgattttt |
| 3181 | atatttgggg catctctata gaaagtgacc accaaggcca gtaagtacgg gaaaaaatgt |
| 3241 | ttactaactt cctcagagat tcgtgatacg cgtttctcca ctgacagaca tttaaaaaca |
| 3301 | accttcagct ccgtttcaat caatcacctc gacttgtttt ttagcatgga cactgccagc |
| 3361 | aggacagaca gggatggagt aaaccgaagt caatttcagg gctcttggcg tgttggacac |
| 3421 | agaagaaatc ctagtgcagc ctttggtagc taacagtcac tgatttlata attggagaat |
| 3481 | gcgtaaagat tcallittca aggagaagag cctgcaaatg gccaatgaag gaggtaaata |
| 3541 | aactaagata ttccgaggga agggacccag gccacctccc ttccgcaggt ctgcagatga |
| 3601 | agggtttttt gaatgaaatg ccactgtgca ttttcagaaa aaaaaatctc tgataaacag |
| 3661 | actttgaatg gatgtttgtt cctcctgatt ctcttttctc ttcgtggcga cttagagttg |
| 3721 | gcggatattc ggaactgtga atgtacatag cgttgagtta aaccccttgt gtgtgagaca |
| 3781 | ggacgcagcg ggcccctggt ggcctggggg ccagacccgt gggcaggtgg ggcatgggcc |
| 3841 | ctggcctgcg gggacctgct ggggtgtgag ggcagaggga gggttgccat gaaggaactt |
| 3901 | gggattttca atggaataag taaaacataa agtctatact tgggaaaaaa aaaaaaaaaa |
| 3961 | aaaaaaaaaa aaaaaaaaaa aaaaaaaaaa aaaa |

SEQ ID NO: 10 LPCAT1 polypeptide sequence NCBI Ref Seq: NP_079106

| | |
|---|---|
| 1 | mrlrgcgpra apassagasd arllappgrn pfvhelrlsa lqkaqvalmt ltlfpvrllv |
| 61 | aaammllawp lalvaslgsa ekepeqppal wrkvvdfllk aimrtmwfag gfhrvavkgr |
| 121 | qalpteaail tlaphssyfd aipvtmtmss ivmkaesrdi piwgtliqyi rpvfvsrsdq |
| 181 | dsrrktveei krraqsngkw pqimifpegt ctnrtclitf kpgafipgap vqpvvlrypn |
| 241 | kldtitwtwq gpgaleilwl tlcqfhnqve ieflpvysps eeekrnpaly asnvrrvmae |
| 301 | algvsvtdyt fedcqlalae gqlrlpadtc llefarlvrg lglkpeklek dldryserar |
| 361 | mkggekigia efaaslevpv sdlledmfsl fdesgsgevd lrecvvalsv vcrpartldt |
| 421 | iqlafkmyga qedgsvgegd lscilktalg vaeltvtdlf raidqeekgk itfadfhrfa |
| 481 | emypafaeey lypdqthfes caetspapip ngfcadfspe nsdagrkpvr kkld |

What is claimed is:

1. A method for antenatally assessing fetal lung maturity, including the steps of:
collecting a sample of maternal serum;
obtaining cell free messenger RNA (mRNA) from the sample of maternal serum;
determining a level of cell free lysophospholipid acyltransferase 1 (LPCAT1) mRNA in the sample of maternal serum;
comparing the level of cell free LPCAT1 mRNA in the sample of maternal serum to a reference standard relating a level of cell free LPCAT1 mRNA to a level of fetal lung maturity;
determining the level of lung maturity of a fetus corresponding to the level of cell free LPCAT1 mRNA in the sample; and assessing the level of lung maturity of a fetus.

2. The method of claim 1, wherein the amount of cell free LPCAT1 mRNA in the sample of maternal serum is further defined as a copy number of cell free LPCAT1 mRNA per cc of amniotic fluid.

3. The method of claim 2, further including, after the step of obtaining cell free mRNA, the step of reverse transcribing the mRNA and quantitating by reverse transcriptase polymerase chain reaction (RT-PCR) the resulting LPCAT1 cDNA amplicon.

4. The method of claim 3, wherein the quantitating step further includes the steps of amplifying the LPCAT1 cDNA with a forward primer including an oligonucleotide with at least 95% identity with SEQ ID NO: 1 or the exact complement thereof, and with a reverse primer including a including an oligonucleotide with least 95% identity with SEQ ID NO: 2 or the exact complement thereof, and quantitating a PCR product including SEQ ID NO: 4 with a fluorescent reporter probe including an oligonucleotide including SEQ ID NO: 3 or the exact complement thereof.

5. A method for assessing the risk of a fetus for developing neonatal respiratory distress syndrome, including the steps of:
collecting a sample of maternal serum;
obtaining cell free messenger RNA (mRNA) from the sample of maternal serum;
determining a level of cell free lysophospholipid acyltransferase 1 (LPCAT1) mRNA in the sample of maternal serum;
comparing the level of cell free LPCAT1 mRNA in the sample of maternal serum to a threshold level of cell free LPCAT1 mRNA, wherein a level of cell free LPCAT1 mRNA below the threshold level indicates risk that a fetus will develop neonatal respiratory distress syndrome; and
determining whether risk a fetus is at risk of neonatal respiratory distress syndrome.

6. The method of claim 5 wherein the level of cell free LPCAT1mRNA in the sample of maternal serum is further defined as a copy number of cell free LPCAT1mRNA per cc of maternal serum.

7. The method of claim 6, wherein the threshold level of cell free LPCAT1mRNA is further defined as 100 copies of cell free LPCAT1mRNA per cc of maternal serum.

8. The method of claim 6, wherein the threshold level of cell free LPCAT1mRNA is further defined as a level of cell free LPCAT1 mRNA known to be correlated to a level of an amniotic fluid biomarker associated with fetal risk for developing neonatal respiratory distress syndrome.

9. The method of claim 8, wherein the amniotic fluid biomarker is further defined as lamellar body count (LBC), and the threshold level of cell free LPCAT1mRNA is further defined as a level of cell free LPCAT1 mRNA known to be correlated with an amniotic fluid LBC of 40,000-50,000 lamellar bodies/μl amniotic fluid.

10. The method of claim 9, wherein the threshold level of cell free LPCAT1 mRNA is further defined as a level of cell free LPCAT1 mRNA known to be correlated with an amniotic fluid LBC of 50,000 lamellar bodies/μl amniotic fluid.

11. The method of claim 5, further including, after the step of obtaining cell free mRNA, the step of reverse transcribing the mRNA and quantitating by reverse transcriptase polymerase chain reaction (RT-PCR) the resulting LPCAT1 cDNA amplicon.

12. The method of claim 11, wherein the quantitating step further includes the steps of amplifying the LPCAT1 cDNA with a forward primer including an oligonucleotide with at least 95% identity with SEQ ID NO: 1 or the exact complement thereof, and with a reverse primer including a including an oligonucleotide with at least 95% identity with SEQ ID NO: 2 or the exact complement thereof, and quantitating a PCR product including SEQ ID NO: 4 with a fluorescent reporter probe including an oligonucleotide including SEQ ID NO: 3 or the exact complement thereof.

13. The method of claim 5, additionally including the steps of:
determining that the copy number of serum fetal LPCAT1 per unit volume of maternal serum is below the threshold level known to indicate sufficient lung maturity; and treating the pregnant mother or the fetus to reduce the likelihood of respiratory distress at parturition.

14. The method of claim 13, wherein the step of treating a pregnant mother or a fetus is further defined as administering corticosteroid treatments to the pregnant mother to accelerate lung maturation.

15. The method of claim 13, wherein the step of treating the pregnant mother or the fetus is further defined as recommending against preterm delivery.

16. The method of claim 13, wherein the step of treating a pregnant mother or a fetus is further defined as administering gene therapy to the fetus.

17. The method of claim 16, wherein the step of administering gene therapy further includes the steps of administering to a fetus a sufficient quantity of an expression vector encoding LPCAT1 and inducing in the lung tissue of a fetus a sufficient expression of LPCAT1 to prevent respiratory distress upon parturition.


SEQUENCE LISTING (10 sequences)

SEQ ID NO: 1 - DNA, 21 bp, Artificial Sequence
  Forward Primer
aggattctcgcaggaaaacag

SEQ ID NO: 2 - DNA, 22 bp, Artificial Sequence
  Reverse Primer
ttgaaggtaattaggcaggtcc

SEQ ID NO: 3 - DNA, 23 bp, Artificial Sequence
  TaqMan Probe
agaaatcaagagacgggcgcagt

SEQ ID NO: 4 - DNA, 107 bp, Homo sapiens
tcaagatcatcagcaatgcctcctgcaccaccaactgcttagcacccctggccaaggtcatccatgacaactttggtatcgtggaaggactcatgaccacagtccat

SEQ ID NO: 5 - DNA, 22 bp, Artificial Sequence
  Forward Primer GAPDH
agcctcaagatcatcagcaatg

SEQ ID NO: 6 - DNA, 23 bp, Artificial Sequence
  Reverse Primer GAPDH
atggactgtggtcatgagtcctt

SEQ ID NO: 7 - DNA, 18 bp, Artificial Sequence
  TaqMan Probe GAPDH
ccaactgcttagcacccc

SEQ ID NO: 8 - DNA, 1008 bp, Homo sapiens
atggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgacgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccaccttcgacgctggggctggcattgccctcaacgaccactttgtcaagctcatttcctggtatgacaacgaatttggctacagcaacagggtggtggacctcatggcccacatggcctccaaggagtaa

SEQ ID NO: 9 - DNA, 3994 bp, Homo sapiens
gcccgctccagccgccgcgcatcctcggcccgcgccccgagacccgcgcccagctagccccggccccgctcggcgccccaggcagctcggctgcgctcgccgcgggacggcgcggccatgaggctgcggggatgcggaccccgggccgcccctgcctccagcgcaggggccagcgacgctcggctgctggcgcccccggggcggaaccccttcgtgcacgagctgcgcctcagcgccctgcagaaggcccaggtggccctcatgacactgacgctcttcccggtccggctcctggttgccgctgccatgatgctgctggcctggcccctcgcacttgtcgcatccctgggctctgcggagaaggaacccgagcagcccccggccctgtggaggaaggttgtggacttcctgctgaaggccatcatgcgcaccatgtggttcgccggcggcttccaccgggtggccgtgaaggggcggcaggcgctgcccaccgaggcggccatcctcacgctcgcgcctcactcgtcctacttcgacgccatccctgtgaccatgacgatgtcctccatcgtgatgaaggcagagagcagagacatcccgatctggggaactctgatccagtatatacggcctgtgttcgtgtcccggtcagaccaggattctcgcaggaaaacagtagaagaaatcaagagacgggcgcagtccaacggaaagtggccacagataatgatttttccagaaggaacttgtacaaacaggacctgcctaattaccttcaaacctggtgcattcatccctggagcgcccgtccagcctgtggttttacgatatccaaataaactggacaccatcacatggacgtggcaaggacctggagcgctggaaatcctgtggctcacgctgtgtcagtttcacaaccaagtggaaatcgagttccttcctgtgtacagcccttctgaggaggagaagaggaaccccgcgctgtatgccagcaacgtgcggcgagtcatggccgaggccttgggtgtctccgtgactgactacacgttcgaggactgccagctggccctggcggaaggacagctccgtctccccgctgacacttgccttttagaatttgccaggctcgtgcggggcctcgggctaaaaccagaaaagcttgaaaaagatctggacagatactcagaaagagccaggatgaagggaggagagaagataggtattgcggagtttgccgcctccctggaagtccccgtttctgacttgctggaagacatgttttcactgttcgacgagagcggcagcggcgaggtggacctgcgagagtgtgtggttgccctgtctgtcgtctgccggccggcccggaccctggacaccatccagctggctttcaagatgtacggagcgcaagaggacggcagcgtcggcgaaggtgacctgtcctgcatcctcaagacggccctgggggtggcagagctcaccgtgaccgacctattccgagccattgaccaagaggagaaggggaagatcacattcgctgacttccacaggtttgcagaaatgtaccctgccttcgcagaggaatacctgtacccggatcagacacatttcgaaagctgtgcagagacctcacctgcgccaatcccaaacggcttctgtgccgatttcagcccggaaaactcagacgctgggcggaagcctgttcgcaagaagctggattaggacccagggttgcggagagacgcggcccctcccgcgtggacatcaccgccatgagcctctttgcgagtgacctctgggctccgctcctcactcctgctgtacaggcactgtcttcagcccgagttccaggggcctcgggggctgtttgtatcttgttcctttgtgaagtgtgttgcagaaccgacgcttactgtgcgagaatcggagggcgcgcacgcggatcccccgcctggcctggaccccgtggggtcaggttccctgccgggcggggggcaccggtgccgccccgtgttctcccacggggccctggtttcgagtctctgtcacagcctcttccggcggcagcgtgcaccgggcgggcctccgtgcacactcagcacacgcctgccacacagcgtgcgcttgcgtgtcactctggcacgaaacctgtctgcctctgtggatccacagcctggcagagccgagccgtcacctgatttttcagtgtttctacctgtgtgctggagctcatgagtattttataaactccatttaggtacttcaggaaacatgcagcattttttaaaaaatgaaaattgtttttctacttcatttttccttttagagtcaaaggatatttatttataggcctttttttttttaatatagaatctgaggctgtttgggctttgacttaaatttccatcaggcctctctccagcaggtaatccctctccttccgctgggtcccctggggaggtgtgaactcaagggcctagccccaaaacactttttctgcttttcttaatccttttccagtcccctctttttttataaacgttggcagtttgatgtttctgtttcggcataacgtaatccatttcactgtagcctaaactccagtccgaggttggatattgttcaaatgagcagggcccgagctggaagcgcaaggcagccgccgccgtgccgctcctcccttgccctcaggccaggtccctgctggaagcggctgcatcttcctgtcagccctggtttccatggtgactggcgtcacgcagccacccgagtatggctgaccttcctgcagagagaggagccgcagtcttttgcttgtggaaggagacgctgggctgtgcggtgcggagggtgatgaggatgtctggtgacagccgtgcggacaccactcctctctgcagcactgcctcccagcgccagggtcgcgggcacatcccactgagagcgggggtcctgccccatcttagagtcaaaggcagaggggcttccaggccctggatggggtattttggtgtcacctgaagtccctctgacatcaccttgtttcatcattttttatgacagaattagaaacccatccttcaagcacaataatcatcacagacttgagtttgcttcctaaagcaaaggctccgggtttgtttggaaaatttttttgatttctgaaatgaattgatttttatatttggggcatctctatagaaagtgaccaccaaggccagtaagtacgggaaaaaatgtttactaacttcctcagagattcgtgatacgcgtttctccactgacagacatttaaaaacaaccttcagctccgtttcaatcaatcacctcgacttgttttttagcatggacactgccagcaggacagacagggatggagtaaaccgaagtcaatttcagggctcttggcgtgttggacacagaagaaatcctagtgcagcctttggtagctaacagtcactgattttataattggagaatgcgtaaagattcatttttcaaggagaagagcctgcaaatggccaatgaaggaggtaaataaactaagatattccgagggaagggacccaggccacctcccttccgcaggtctgcagatgaagggttttttgaatgaaatgccactgtgcattttcagaaaaaaaaatctctgataaacagactttgaatggatgtttgttcctcctgattctcttttctcttcgtggcgacttagagttggcggatattcggaactgtgaatgtacatagcgttgagttaaaccccttgtgtgtgagacaggacgcagcgggcccctggtggcctgggggccagacccgtgggcaggtggggcatgggccctggcctgcggggacctgctggggtgtgagggcagagggagggttgccatgaaggaacttgggattttcaatggaataagtaaaacataaagtctatacttgggaaaaaaaaaaaaaaaaaaaaaaaaaaaaaaaaaaaaaaaaaaaaaaaaaa

SEQ ID NO: 10 - protein, 534 aa, Homo sapiens
MRLRGCGPRAAPASSAGASDARLLAPPGRNPFVHELRLSALQKAQVALMTLTLFPVRLLVAAAMMLLAWPLALVASLGSAEKEPEQPPALWRKVVDFLLKAIMRTMWFAGGFHRVAVKGRQALPTEAAILTLAPHSSYFDAIPVTMTMSSIVMKAESRDIPIWGTLIQYIRPVFVSRSDQDSRRKTVEEIKRRAQSNGKWPQIMIFPEGTCTNRTCLITFKPGAFIPGAPVQPVVLRYPNKLDTITWTWQGPGALEILWLTLCQFHNQVEIEFLPVYSPSEEEKRNPALYASNVRRVMAEALGVSVTDYTFEDCQLALAEGQLRLPADTCLLEFARLVRGLGLKPEKLEKDLDRYSERARMKGGEKIGIAEFAASLEVPVSDLLEDMFSLFDESGSGEVDLRECVVALSVVCRPARTLDTIQLAFKMYGAQEDGSVGEGDLSCILKTALGVAELTVTDLFRAIDQEEKGKITFADFHRFAEMYPAFAEEYLYPDQTHFESCAETSPAPIPNGFCADFSPENSDAGRKPVRKKLD